(19) United States
(12) Patent Application Publication
(10) Pub. No.: US 2003/0143541 A1
(43) Pub. Date: Jul. 31, 2003

(54) Human pheromone receptors

(76) Inventor: Lakshmi Sangameswaran, San Jose, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/032,697

(22) Filed: Dec. 27, 2001

(51) Int. Cl. C12Q 1/68; G01N 33/53; C07H 21/04; G01N 33/567; C12P 21/02; C12N 5/06; C07K 14/705

(52) U.S. Cl. 435/6; 435/69.1; 435/320.1; 435/325; 530/350; 536/23.2; 514/44; 435/7.2; 800/8

(57) ABSTRACT

The present invention relates to mammalian receptors, particularly human pheromone receptors and the expression of these receptors in selected cells and tissues. More specifically, the invention encompasses novel human vomeronasal organ (VNO) receptors. The invention contemplates nucleic acid molecules and fragments thereof encoding human pheromone receptors and agents that specifically bind to the polypeptides comprising human pheromone receptors. The invention also encompasses vectors and cells for producing the nucleic acid molecules encoding human pheromone receptors as well as vectors and cells for expressing receptor polypeptides. The invention further relates to methods for isolation, characterization and detection of the nucleic acid molecules, polypeptides, and agents that bind to the receptors. The invention also encompasses diagnosis and treatment of diseases or disorders related to the aberrant expression of the human pheromone receptors hV3R1 and hV3R8.

### BACKGROUND OF THE INVENTION

a) Field of the Invention

The present invention relates to mammalian receptors, particularly human pheromone receptors and the expression of these receptors in selected cells and tissues. The invention further relates to methods of nucleic acid detection.

b) Description of Related Art

The Use of Bioinformatics to Identify Biological Molecules

Computer software and electronic databases have become invaluable for the analysis of biological information. Bioinformatics is the application of computer science to the interpretation and management of biological data. Bioinformatics is used to identify genes from sequenced regions of DNA, predict protein structure and function, determine metabolic pathways, evaluate protein sites for drug targeting, and analyze transcription and proteomic data.

The primary influx of information is in the form of raw DNA sequences which are sorted by national and international organizations. Such organizations are the National Centre for Biotechnology Information (NCBI) in the US, and the European Bioinformatics Institute (EBI) in the UK. These two organizations sort and annotate incoming DNA data. Such data is then released in the form of Genbank and EMBL databases. Major releases of the databases take place every few months while incremental updates are released daily. Each of these databases consists of a number of sections, wherein each section contains up to 100,000 sequences. The sections are essentially phylogenetic, such as phage, rodent, human, and the like. Further, the sections describe the type of data such as EST (expressed sequence tags) and STS (sequence tagged sites), etc. Each entry, uniquely identified by a stable accession number and a potentially changing identifier, consists of a number of fields for keywords. This information is sorted, collated, and indexed. The nucleotide databases can then be converted to protein databases. In addition, there are also separate protein databases such as the Swissprot database, maintained in Switzerland and at the EBI. Each entry in Swissprot is fully annotated, and a sequence is usually not entered into that database unless there is supporting literature. This makes for a highly reliable and relatively small (˜80,000 sequences) protein data set. The remainder of the protein sequences end up in a database called the TrEMBL database. There is also a database called Genpept which is produced by parsing the corresponding GenBank release for translated coding regions of GenBank sequences. As the numbers of genome sequencing projects are constantly increasing, more specialized databases restricted to individual organisms are being developed. Model organism databases exist for many species, including Yeast, Drosophila, Mouse, Human, and the like. Other databases store more specialized data such as protein secondary structure, folds, and domains (e.g., PROSITE (Functional motifs database), PDB (Brookhaven Protein Structure Database), etc.). Moreover, there are countless companies that maintain private databases with proprietary biological data, wherein access is restricted to selected customers.

The database user may employ specific query strategies (e.g., BLAST, aligning, etc.) as well as sophisticated tools (e.g., proprietary algorithms, unique software, etc.) for nucleic acid and protein searches when using any number of existing private and public databases. Querying such databases effectively is not a trivial task. For example, to align two sequences of 100 amino acids, there are about 10E⁷⁵ ways in which they can be aligned onto each other (using gaps in one or the other sequences). The optimal global solution is usually that which minimizes the number of gaps and maximizes the number of matches across the complete lengths of both sequences. Although this may mask shorter local regions of more significance, methods are capable of detecting both. It is useful to use biological scoring, wherein biological information is used to consider methods of scoring. For example, Cys-Cys matches may be considered more relevant than Ala-Ala matches. It is further useful to distinguish between gap insertion and gap extension penalties. The mere introduction of a gap is biologically more important than the actual length of a gap. In addition the output should be amenable to statistical assessment because the probability for a given alignment should be statistically measurable.

Two known methods of obtaining computer alignments are DotPlots, and Dynamic Programming Methods (e.g., Smith & Waterman Alignment Method). DotPlots use a computer to visually present a matrix of two sequences. A dot is drawn onto the screen where a match is found between two sequences. Usually, a form of filtering (e.g., word matching, window/stringency filtering, scoring matrix filtering, etc.) is applied. The Smith & Waterman Alignment Method finds the optimal path through a matrix aligning two sequences. The original method, which determines global alignments, was developed by Needleman and Munch and later modified by Smith and Waterman to determine local alignments, wherein many modifications to this method are known in the art. In addition to different computer alignment methods, there are also different database searching methods. Examples of well known searching methods are Smith & Waterman type searches, FASTA, and BLAST.

One aim of database searching is to identify a given query sequence. This is an invaluable step prior to cloning experiments, often simply to exclude contamination. For a human sequence, there is about a 50% chance that an EST exists for a given gene. For a bacterial or yeast sequence, there is a nearly 70% chance that an identified gene is known. It is also possible to identify multiple extended families across organisms, which implies conservation of evolution, hence, conservation in structure. A pairwise alignment provides a measure of percentage identity and similarity. This can be expressed as a threshold on the scoring matrix. Any similarity of better than 25% identity may be suggestive of homology, i.e., conservation of structure (Hugh Salter, http://www.molbiol.ox.ac.uk/tutorials/year2_theory.html). Clearly, biological databases have become an invaluable tool in identifying individual genes and gene families as well predicting protein structure and function.

The Vomeronasal Organ and Related Receptors

Small, volatile and non-volatile organic molecules, commonly referred to as pheromones, mediate chemical communication between animals and may do so in a manner specific to a species (species-specific). Pheromones are present in the secretions and excretions of various organs and tissues, including the skin, and represent diverse families of chemical structures. Pheromones are known to or implicated to play essential roles in sexual activity, reproductive biology, and other innate animal behaviors (Luscher et al., (1959) Nature 18:55-56; Meredith (1983) Pheromones and Reproduction in Mammals (Vandenbergh, ed.) pp. 199-252, Academic Press; Stem et al., (1998) Nature 392:177-179; Wysocki, (1979) Neurosci. Biobehav. Rev. 3:301-341; Jacob et al., (2000) Hormones and Behavior 37:57-78; Grosser et al., (2000) Psychoneuroendocrinology 25:289-299).

Some naturally occurring pheromones are detected by the vomeronasal organ (VNO) in animals. In humans, the VNO is also known as Jacobson's organ. In animals, including humans, the VNO is a small dead-end tubular structure with an opening into the nasal cavity, located bilaterally at the base of the nasal septum (Berliner, (1996) J. Steroid Biochem. Molec. Biol. 58:1-2; Gaafar et al., (1998) Acta Otolargyngol. 118:408-412; Moran et al., (1991) J. Steroid Biochem. Molec. Biol. 39:545-552), Smith et al., (1998) Micro. Res. Tech. 41:483-491). Signals from the VNO are transmitted through the accessory olfactory bulb to the amygdala and hypothalamus (Broadwell et al., (1975) J. Comp. Neurol. 163:329-346; Kevetter et al., (1981) J. Comp. Neurol. 197:81-98). Furthermore, pheromones may be detected by other tissues or cells. For example, although goldfish do not have a defined VNO, pheromones are detected in this species by a tissue having both olfactory and pheromone receptors. Cao et al. ((1998) Proc. Natl. Acad. Sci. USA 95:11987-11992) have successfully isolated homologues from a goldfish cDNA library using probes based on the rodent VNO receptor sequences.

Immunohistochemical staining of adult human VNO epithelium detects neuron-specific enolase and polypeptide gene product 9.5 (PGP 9.5). Both of these proteins are neuronal and neuroendocrine markers in some bipolar cells with morphological similarities to olfactory receptor neurons (Takami et al., (1993) Neuroreport 4:375-378). Interestingly, Takami et al. did not detect olfactory marker polypeptide (OMP) in the human VNO, even though it is expressed in the VNO of other animals, including rodents. This may reflect an important difference among species. More recent findings known in the art show that the majority of the cells lining the lumen of the human VNO stain with antibodies to synaptophysin and/or chromogranin which are also markers for neuronal and/or neuroendocrine cells, respectively.

It is well known in the art that surgical ablation of the VNO in male rodents alters a variety of endocrine-mediated responses to female pheromones, including androgen surges, vocalization, territorial marking, and inter-male aggression. Ablation of the VNO in female rodents delays or prevents activation of reproduction, abolishes the effects of over-crowding on sexual maturation, and reduces maternal responses to intruders (Wysocki et al., (1991) J. Steroid Biochem. Molec. Biol. 39:661-669). In humans, the defect(s) that cause(s) the inherited hypogonadal disorder, Kallmann Syndrome, is associated with defective development of the VNO-terminalis complex (Kallmann et al., (1943) Am. J Ment. Defic. 48:203-236). Thus, these results suggest that pheromone receptors or their absence mediate physiological (e.g., pathophysiological) and behavioral effects in animals, including humans.

Similarly, researchers have studied the brain activity of humans in response to specific pheromones. Following the delivery of the putative pheromone estra-1,3,5(10), 16-tetraen-3-yl acetate (PH15) to human volunteers, the brain activity of the volunteers was studied using functional magnetic resonance imaging (fMRI). The studies detected a dose-dependent activation of the anterior medial thalamus, inferior frontal gyrus and other regions of the brain, in the absence of detectable odor. Thus, this fMRI data supports the existence of a functional neurological connection between pheromone receptors and the human brain (Savic et al., vide infra). Using fMRI methods, it was also demonstrated that in women androgen-like compounds activate the hypothalamus in the preoptic and ventromedial nuclei, while in men, estrogen-like substances activate the hypothalamus in the paraventricular and dorsomedial nuclei. The study concluded that the preferential sex-associated hypothalamic activation suggests a potential physiological substrate for sex-differentiated behavioral responses in humans (Savic et al., (2001) Neuron IEP (Published Online) Aug.3, 2001).

Naturally occurring pheromones (e.g., estra-1,3,5(10), 16-tetraen-3-ol and androsta-4,16-dien-3-one) delivered to human subjects may induce bradycardia, bradypnea, the increase of core body temperature and other physiological responses in the subjects (Stem et al. (1998) Nature March 12; 392(6672):177-9). In addition, it was demonstrated that odorless human pheromones, obtained from the axillae of women at different stages of the menstrual cycle, exert opposing effects on ovulation when applied above the lips where they can volatilize into the nasal cavity of the recipient females. Some pheromones are sexually dimorphic in that they act specifically or differentially in females or in males, and other pheromones exert particular effects (e.g., opposite to normal function) on autonomic reflexes such as body temperature (supra, Stem et al.). Hence, these data indicate that pheromones are capable of exerting physiological effects in vivo.

The delivery of only femtomole quantities of any of the several existing proprietary, synthetic pheromones to human volunteers rapidly induced reproducible and negative voltage potentials, characteristic of mass receptor potentials (Berliner et al., Monti-Bloch et al., Grosser et al., vide infra). In this study, the reproducible and negative voltage potentials were measured locally with a multifunctional probe. It was concluded that the magnitude of the response may be dependent on the dose of pheromone applied or administered, and may be accompanied by changes in the function of the autonomic nervous system, brain wave activity, gonadotropin secretion, and/or mood (Berliner et al., (1996) J Steroid Biochem, Molec. Biol. 58:259-265; (1998a) J. Steroid Biochem. Molec. Biol. 65:237-242; Monti-Bloch et al., (1998b) Ann. N.Y. Acad. Sci. 855:373-389; Monti-Bloch et al., (1994) Pyschoneuroendocrinology 19:673-686; Monti-Bloch et al., (1991) J. Steroid Biochem. Molec. Biol. 39:573-582; Grosser et al., (2000) Psychoneuroendocrinology 25:289-299). Thus, the effect of pheromones, in vivo or in vitro, may be demonstrated by detecting, measuring and monitoring physiological and/or behavioral responses.

The rodent VNO has been shown to be associated with G protein-coupled receptors. cDNAs of rodent VNO receptors that are specifically expressed in the VNO have been cloned. The sequences of the cloned rodent receptor cDNAs indicate that these receptors belong to the superfamily of G protein-coupled receptors containing seven transmembrane domains. However, the sequence of the cloned rodent receptors are unrelated to any of the sequences of the G protein-coupled receptors expressed in the olfactory epithelium (Dulac et al., (1995) Cell 83:495-206; Herrada et al., (1997) Cell 90:763-773; Matsunami et al., (1997) Cell 90:775-784; Ryba et al., (1997) Neuron 19:371-379; Saito et al., (1998) Brain Res. Molec. Brain Res. 60:215-227; Pantages et al. (2000) Neuron 28:835-845). In some of the clones, database comparisons identified motifs common to Ca²⁺-sensing receptors and metabotropic glutamate receptors. In addition, each cloned rodent receptor messenger RNA (mRNA) was detected by in situ hybridization in only a small number of neuroepithelial cells that are dispersed throughout the rodent VNO and, thus, may be specifically or differentially expressed by subpopulations of neuroepithelial cells (supra, Dulac et al., 1995; Herrada et al., 1997; Matsunami et al., 1997; Ryba et al., 1997; Saito et al., 1998).

The cloned rodent VNO receptors were assigned to three different multi-gene families, V1R, V2R or V3R, based on the following criteria: (i) the length of the extracellular (N-terminal) polypeptide domain; (ii) sequence homology and (iii) the isoform of the signal-transducing G protein co-expressed in the same cell. The rodent receptors in the V1R family have a relatively short extracellular N-terminal domain and are expressed primarily in cells that express a Gαᵢ₂ isoform of G protein. The rodent receptors in the V2R family have a long extracellular N-terminal domain and are expressed primarily in cells that express a Gα₀ isoform of G protein. Differences at the N-terminus between the families may reflect differences in the structure of the receptor ligand and/or in the location of the ligand-binding domain of the receptor (supra, Matsunami et al., 1997; Ryba et al., 1997; and Krieger et al., (1999) J. Biol. Chem. 274:4656-4662). Neuroepithelial cells expressing these distinct G protein isoforms were found to be spatially segregated in the VNO in separate apical and basal longitudinal zones, suggesting that the differences between the rodent receptor families are physiologically significant. Krieger et al. (1999, supra) have also shown that G protein-coupled receptors expressed in the rodent VNO are functionally linked to signal transduction pathways. Their results indicate that volatile and non-volatile pheromonal components of male rat urine selectively activate the major Gα protein subtypes (Gᵢ and G₀, respectively) expressed in the VNO of female rats. Thus, these data imply that the rodent receptors of the V1R family, which are co-expressed with Gᵢ, respond to volatile compounds whereas the rodent receptors of the V2R family, which are co-expressed with G₀, respond to non-volatile compounds, for example, non-volatile polypeptide components of the urine.

Dulac et al. (1995, supra) have estimated that, in total, the rat V1R family contains approximately 35 candidate pheromone receptors. Herrada et al. (1997, supra) and Ryba et al. (1997) have estimated that the rat V2R family contains an additional 100 candidate pheromone receptors. Of the various rodent tissues tested, only mRNA from the VNO gave a positive signal on Northern blots probed with the ³²P-labeled cloned rodent VNO receptor cDNAs. Thus, these results suggest that some novel G protein-coupled receptors may be specifically expressed in the VNO. However, pheromone receptors may be specifically or differentially expressed in other tissues and may be expressed in more than one type of tissue. For example, Rodriguez et al. isolated a human homologue of a rodent pheromone receptor that is expressed in several types of human tissues, including olfactory epithelium (Rodriguez et al., Nature Genetics 26:18-19; 2000). In addition, a pheromone may be recognized by a specific pheromone receptor or by a combination of different pheromone receptors.

It has been shown that at reduced stringency conditions for nucleic acid hybridization, the cloned rodent VNO receptor cDNAs cross-hybridize to human genomic DNA. Dulac et al. (1995, supra) detected approximately 15 human homologues that cross-hybridize to probes prepared from the rat receptors of the V1R family. Herrada et al. (1997, supra) detected an additional 10 human homologues that cross-hybridize to probes prepared from rat receptors of the V2R family. Two genomic DNA clones encoding human homologues of rodent receptors of the V1R family have been isolated and sequenced. The receptor polypeptide sequence encoded by these two genomic DNA clones have been shown to have approximately 40-50% sequence identity with the polypeptide sequence of a rodent receptor of the V1R family. However, both human genomic clones have a stop codon in the putative pheromone receptor polypeptide sequence and may therefore be pseudogenes (Dulac et al., supra). Nevertheless, cross-hybridization of the rodent VNO receptor probes to the human genomic DNA suggests that there is evolutionary conservation of G protein-coupled pheromone receptors.

### SUMMARY OF THE INVENTION

The present invention relates to mammalian receptors, particularly human pheromone receptors and the expression of these receptors in selected cells and tissues. More specifically, the invention encompasses novel human vomeronasal organ (VNO) receptors. The invention further relates to methods of nucleic acid detection and high throughput drug screening assays.

One aspect of the invention provides for an isolated nucleic acid molecule comprising a nucleotide sequence encoding a polypeptide of SEQ ID NO: 2, or polypeptide fragment thereof, wherein the polypeptide or polypeptide fragment mediates a physiological and/or behavioral effect in mammals. Particularly, the nucleic acid molecule comprises a nucleotide sequence encoding the human pheromone receptor, hV3R1. In one embodiment, the nucleic acid molecule comprises the sequence as set forth in SEQ ID NO: 1. More specifically, the nucleic acid molecule may be a DNA or RNA derived from human tissue, cells or cell lines or a recombinant DNA. Still more specifically, the nucleic acid molecule may be a genomic DNA, cDNA, or mRNA.

Another aspect of the invention provides for an isolated nucleic acid molecule comprising a nucleotide sequence encoding a polypeptide of SEQ ID NO: 4, or polypeptide fragment thereof, wherein the polypeptide or polypeptide fragment mediates a physiological and/or behavioral effect in mammals. Particularly, the nucleic acid molecule comprises a nucleotide sequence encoding the human pheromone receptor, hV3R8. In one embodiment, the nucleic acid molecule comprises the sequence as set forth in SEQ ID NO: 3. More specifically, the nucleic acid molecule may be a DNA or RNA derived from human tissue, cells or cell lines or a recombinant DNA. Still more specifically, the nucleic acid molecule may be a genomic DNA, cDNA, or mRNA.

Another aspect of the invention provides a method for mediating a physiological disorder comprising of and administering an effective amount of an isolated polypeptide, or polypeptide fragment thereof, to a subject in need of treatment for diseases and disorders including, but not limited to, infertility; disorders related to contraception; disorders related to hormonal regulation, sexual dysfunction, and erectile dysfunction; neurological-, psychiatric-, liver- and spleen disorders; cardiovascular disorders; cancer; and known or unknown disorders related to the aberrant expression of the human pheromone receptors hV3R1 and/or hV3R8. Alternatively, the method may include administering a composition which includes an isolated polyclonal or monoclonal antibody that specifically binds to an isolated polypeptide, or fragment thereof. The method may further include administering to the subject an effective amount of the composition by topical, oral, inhalable, subcutaneous or intramuscular administration.

The invention further contemplates a method for detecting a target nucleic acid molecule or polypeptide in a sample by using a probe.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The present invention is best understood when read in conjunction with the accompanying figures that serve to illustrate the preferred embodiments. It is understood, however, that the invention is not limited to the specific embodiments disclosed in the figures.

FIG. 1 provides a schematic representation of the methods employed in identifying novel vomeronasal organ (VNO) receptors, including a summary for the workflow of performed experiments.

FIG. 2 depicts the nucleic acid and amino acid sequence of the novel VNO receptor hV3R1.

FIG. 3 depicts the nucleic acid and amino acid sequence of the novel VNO receptor hV3R8.

### DETAILED DESCRIPTION OF THE INVENTION

a) Definitions and General Parameters

The following definitions are set forth to illustrate and define the meaning and scope of the various terms used to describe the invention herein. As used herein, the singular forms “a,” “an” and “the” include plural reference unless the context clearly dictates otherwise. Further, unless defined otherwise, all technical and scientific terms used herein have the same meaning as commonly understood by one of ordinary skill in the art to which this invention pertains.

The term “polypeptide” means a poly(amino acid) comprising at least two amino acids linked by peptide bonds. A “protein” is a polypeptide which is encoded by a gene.

A “ligand” is a molecule that specifically binds to another molecule. An example of a ligand is a pheromone that specifically binds to a pheromone receptor. Another example of a ligand is an antibody that specifically binds to a pheromone receptor. In yet another example, an antibody may bind to a specific ligand. As used herein, the term “ligand” and “agent” may be used interchangeably herein.

A “pheromone” functions as a chemosensory messenger; specifically binds to a pheromone receptor or to cells or tissue in which the pheromone receptor is expressed, and/or modulates the function, expression and/or activity of the receptor, and/or modulates a physiological or behavioral effect mediated by the receptor. The specific binding and/or modulation by a pheromone may be specific to a species and/or sexually dimorphic. For example, human pheromones may specifically or differentially bind to human pheromone receptors and/or modulate a physiological or behavioral effect in humans, and not other species. Also, for example, the binding and/or modulation may be specific to males or may be specific to females. This binding and/or modulation may be detected, for example, by measuring the change in the electrical potential of neuroepithelial tissue in the presence of the pheromone. Human pheromones may induce a change in human neuroepithelial tissue of the appropriate sex, for example, the change in total electrical potential is at least about -5 millivolts. In humans, as well as other animals, the binding of pheromones to pheromone receptors may be sexually dimorphic and/or modulate sexually dimorphic changes in receptor binding potential, in vivo. Pheromones may be isolated or synthetic compounds, or modifications of isolated or synthetic compounds. Human pheromones may be extracted and purified, for example, from human skin or may be synthesized, and may specifically or differentially bind to human pheromone receptors, cells or tissues. Such pheromones may mediate a physiological and/or behavioral effect in mammals. For example, such pheromones may mediate development, reproduction and related behaviors.

As used herein, “VNO receptors” are receptors expressed in the vomeronasal organ (VNO), and include some pheromone receptors. However, pheromone receptors may be expressed in tissues other than the VNO.

As used herein, “agents” are any substance from any source which functions as a chemosensory messenger that specifically binds to a pheromone receptor expressed in the VNO or expressed in other tissue, and/or modulates a physiological or behavioral effect mediated by the pheromone receptor. More particularly, the agent may modulate the expression, function and/or activity of a pheromone receptor. An example of an agent is a ligand such as a pheromone that specifically binds to a pheromone receptor. Another example of an agent is an antibody that specifically binds to the pheromone receptor or pheromone receptor ligand.

As used herein, an “antibody” refers to a protein consisting of one or more polypeptides substantially encoded by immunoglobulin genes or fragments of immunoglobulin genes. Antibodies may exist as intact immunoglobulins or as a number of fragments, including those well-characterized fragments produced by digestion with various peptidases. While various antibody fragments are defined in terms of the digestion of an intact antibody, one of skill will appreciate that antibody fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Furthermore, an “antibody” refers to an immunoglobulin that specifically binds to an antigen. Pheromones that bind to a human VNO receptor are examples of antigens. The antibody may be monoclonal or polyclonal and can be prepared by techniques that are well known in the art such as immunization of a host and collection of polyclonal antibody sera therefrom or preparation of hybridoma cell lines and collection of monoclonal antibody secreted therefrom. Further involved may be the cloning and expression of nucleotide antibody sequences, or mutagenized or modified versions thereof, coding for amino acid sequences required for specific binding of the antibody to the receptor or fragment thereof. Antibodies may include a complete or full-length immunoglobulin, or fragment thereof. Such immunoglobulins include, but are not limited to, the various classes and isotypes, such as IgA, IgD, IgE, IgGI, IgG2a, IgG2b and IgG3, IgM. Antibody fragments encompassed by the use of the term “antibodies” include, but are not limited to, Fab, Fab′, F(ab′)₂, scFv, Fv, dsFv diabody, and Fd fragments. In addition, aggregates, polymers, and conjugates of immunoglobulins or fragments thereof can be used where the antibody specifically binds to a receptor or fragment thereof. Further, humanized or chimeric monoclonal antibodies may be used. Also, phage display libraries may be used to screen for monoclonal antibodies with a particular and/or specific binding affinity for an antigen.

A “polynucleotide”, “oligonucleotide”, or “nucleic acid” includes, but is not limited to, mRNA, cDNA, genomic DNA, and synthetic DNA and RNA sequences, comprising the natural nucleoside bases adenine, guanine, cytosine, thymine, and uracil. The term also encompasses sequences having one or more modified nucleosides. The terms “polynucleotide” and “oligonucleotide” are used interchangeably herein. No limitation as to length or to synthetic origin are suggested by the use of either of these terms herein.

A “cDNA” refers to complementary DNA that is synthesized from an mRNA template that corresponds to expressed sequences of genomic DNA.

An “oligonucleotide probe” is a nucleic acid molecule, for example a DNA or RNA, that includes a sufficient number of nucleotides to hybridize specifically to another nucleic acid molecule under high or reduced stringency nucleic acid hybridization conditions. Reduced stringency means at a stringency less than high stringency defined herein (vide infra). The oligonucleotide probe can specifically hybridize to a DNA or RNA having a complementary, homologous, or related sequence. An oligonucleotide probe may contain any number of nucleotides. For example, the oligonucleotide probe may contain as few as 7 nucleotides and as many as is desired, and preferably about 7 to about 50 nucleotides. The conditions and protocols for such hybridization are well known to those of skill in the art, as are the effects of probe length, temperature, degree of mismatch, salt concentration and other parameters of the hybridization reaction. For example, the lower the temperature and higher the salt concentration at which the hybridization reaction is carried out, the greater the degree of mismatch that may be present between the hybridized molecules.

An oligonucleotide probe may include a “detectable label”, for example, ³⁵S, ³²p, ³H and ¹⁴C. The oligonucleotide probe may be labeled, for example, by nick-translation in the presence of deoxyuridylate triphosphate biotinylated at the 5′-position of the uracil moiety. The resulting probe has incorporated the biotinylated uridylate in place of thymidylate and can be detected based on the binding of streptavidin to the biotin moiety. Any other detectable label may be used. The methods for labeling a nucleic acid or oligonucleotide probe with a detectable label are well known in the art.

“Stringency,” as used herein, refers to the stringency of nucleic acid hybridization. “High stringency,” refers to 0.1×SSPE (1×SSPE=150 mM NaCl, 10 mM NaH₂PO₄, pH 7.4, 1 mM EDTA), 0.1% SDS, 65° C. “Medium stringency” refers to 0.2×SSPE, 0.1% SDS, 50° C. “Low stringency” refers to 1.0×SSPE, 0.1% SDS, 50° C. Any other combination of salt, temperature and other reagents that results in the same or similar degree of nucleic acid hybridization as the above hybridization conditions may be used. “Reduced stringency” refers to a stringency of nucleic hybridization that is lower than high stringency defined herein. The term “Stringency”, as used herein, may also be used to refer to a term used in Bioinformatics where nucleic acid sequences are identified via the computer. When a statistic or score is calculated for a window of residues (e.g., nucleotides), the term stringency is used to refer to the minimum score which will be used to identify a match. For example, with a window of 10 and a stringency of 6, 6 out of 10 bases must be identical in order for a match to exist. There are many different Bioinformatics approaches that allow for the identification and comparison of nucleic acid sequences and homologies, no limitation is suggested herein.

“Vector,” “expression construct” or “expression vector” as used herein refers to a DNA or RNA containing a site for inserting a nucleic acid molecule and operably linking the inserted nucleic acid molecule (insert) to the vector so that the insert may be replicated, and/or a polypeptide encoded by the insert may be expressed in vitro or in vivo. The vector is a polynucleotide comprised of a single strand, double strand, or circular DNA or RNA. The insert may be, for example a DNA or RNA, and may encode a polypeptide. A “vector” is comprised of the following elements operatively linked at appropriate distances for allowing functional gene expression: replication origin, promoter, enhancer, 5′ mRNA leader sequence, ribosomal binding site, nucleic acid cassette, termination and polyadenylation sites, and selectable marker sequences. One or more of these elements may be omitted in specific applications. The nucleic acid cassette can include a restriction site for insertion of the nucleic acid sequence to be expressed. In a functional vector the nucleic acid cassette contains the nucleic acid sequence to be expressed including translation initiation and termination sites. An expression vector is constructed so that the particular coding sequence is located in the vector with the appropriate regulatory sequences, the positioning and orientation of the coding sequence with respect to the control sequences being such that the coding sequence is transcribed under the “control” of the control sequences. Modification of the sequences encoding the particular protein of interest may be desirable to achieve this end. For example, in some cases it may be necessary to modify the sequence so that it may be attached to the control sequences with the appropriate orientation; or to maintain the reading frame. The control sequences and other regulatory sequences may be ligated to the coding sequence prior to insertion into a vector. Alternatively, the coding sequence can be cloned directly into an expression vector which already contains the control sequences and an appropriate restriction site which is in reading frame with and under regulatory control of the control sequences. In addition, the insert or vector may contain an intron that is preferably ≧100 base pairs (bp). A vector may also be a plasmid as described herein.

A “plasmid” refers to a circular or supercoiled DNA or RNA capable of replicating in a host cell. A plasmid may also be a vector, as described herein (supra).

“Fragment” refers to a nucleic acid molecule that encodes less than the full-length amino acid sequence of an isolated polypeptide; or a polypeptide containing less than the full-length amino acid sequence of an isolated polypeptide. Examples of isolated polypeptides are human pheromone receptors and antibodies.

“Expression profile” refers to the pattern of expression of a polypeptide or nucleic acid molecule, for example a gene, in vivo or in vitro. The pattern of expression of the polypeptide or nucleic acid molecule encoding the polypeptide may be detected, for example, in specific cells or tissues, and/or under specific conditions or circumstances. The nucleic acid molecule may be, for example, DNA or RNA.

“Modulation” as used herein with reference to pheromone receptors, refers to the induction or increase, or inhibition or decrease, or other change in the expression, function and/or activity of the receptors, and/or physiological or behavioral effect mediated by the receptors, as detected by the methods described herein or well known in the art.

“Sexually dimorphic” refers to an effect of a compound or composition that is specific to males or specific to females of the same species. For example, an effect of a pheromone may be specific to males or to females of the same species or the effect may elicit the opposite effect in male versus female.

b) Polynucleotides and Polypeptides of the Human Pheromone Receptors hV3R1 and hV3R8

The materials provided by the invention include: isolated, modified and synthetic nucleic acid molecules encoding mammalian pheromone receptors and the encoded receptor polypeptides, and fragments of such nucleic acid molecules and polypeptides, that mediate a physiological and/or behavioral effect in mammals; antibodies that specifically bind to the polypeptides and polypeptide fragments; vectors suitable for the in vitro or in vivo expression of the nucleic acid molecules and polypeptides of the present invention; and mammalian, bacterial, insect and yeast cells transduced or transfected with such vectors that stably or transiently express the nucleic acid molecules and polypeptides in vitro or in vivo. Especially useful are mammalian cells transduced or transfected with such vectors, that express the receptors as a cell-surface polypeptide. Such mammalian cells are especially useful for screening for agents that specifically bind to the receptor. More particularly, the mammalian cells are useful for screening for native or synthetic agonists and antagonists that bind to the receptors and modulate the expression, function or activity of the receptors. Also provided are nucleic acid molecules that hybridize to and/or have a nucleotide sequence complementary to the nucleotide sequence of the nucleic acid molecules encoding the receptors. Such nucleic acid molecules are useful as nucleic acid hybridization probes, antisense oligonucleotides and compositions.

The invention provides methods for isolating and expressing the nucleic acid molecules, encoded receptors and fragments thereof, as well as methods of screening for agents that specifically bind to the receptors and/or modulate the activity of the receptors. The invention further provides methods for the detection of the nucleic acid molecules and encoded receptors in a sample, and methods for delivering the compositions to subjects in need of treatment for diseases or disorders including, but not limited to, infertility; disorders related to contraception; disorders related to hormonal regulation, sexual dysfunction, and erectile dysfunction; neurological-, psychiatric-, liver- and spleen disorders; cardiovascular disorders; cancer; and known or unknown disorders related to the aberrant expression of human pheromone receptor(s). Also provided are kits for detecting the nucleic acid molecules and receptors.

One aspect of the invention is an isolated nucleic acid molecule comprising a nucleotide sequence encoding a polypeptide of SEQ ID NO: 2, or polypeptide fragment thereof, wherein the polypeptide or polypeptide fragment mediates a physiological and/or behavioral effect in mammals. Particularly, the nucleic acid molecule comprises a nucleotide sequence encoding the human pheromone receptor, hV3R1. In one embodiment, the nucleic acid molecule comprises the sequence as set forth in SEQ ID NO: 1. More specifically, the nucleic acid molecule may be a DNA or RNA derived from human tissue, cells or cell lines or a recombinant DNA. Still more specifically, the nucleic acid molecule may be a genomic DNA, cDNA, or mRNA.

In another aspect there is provided an isolated nucleic acid molecule that hybridizes under high, medium or low stringency conditions to a nucleic acid encoding a polypeptide of SEQ ID NO: 2, wherein the polypeptide mediates a physiological and/or behavioral effect in mammals. Preferably, the polypeptide mediates a physiological and/or behavioral effect in humans. In a preferred embodiment, the isolated nucleic acid molecule is a DNA or RNA oligonucleotide probe, wherein the oligonucleotide probe is about 7 to about 50 nucleotides in length and, optionally, comprises a detectable label.

In another aspect there is provided a vector comprising a nucleotide sequence encoding a polypeptide of SEQ ID NO: 2, or polypeptide fragment thereof, wherein the polypeptide or polypeptide fragment mediates a physiological and/or behavioral effect in mammals. The polypeptide may be expressed in mammalian-, bacterial-, insect- or yeast cells that are stably transduced or transiently transfected with the vector. In a preferred embodiment, the polypeptide is expressed as a cell-surface receptor, including a functional or partially functional receptor.

In another aspect there is provided an isolated polypeptide of SEQ ID NO: 2, and analogs and fragments of the isolated polypeptide, wherein the polypeptide or polypeptide fragment mediates a physiological and/or behavioral effect in mammals. More particularly, the amino acid sequence of the isolated polypeptide comprises SEQ ID NO: 2.

One aspect of the invention is an isolated nucleic acid molecule comprising a nucleotide sequence encoding a polypeptide of SEQ ID NO: 4, or polypeptide fragment thereof, wherein the polypeptide or polypeptide fragment mediates a physiological and/or behavioral effect in mammals. Particularly, the nucleic acid molecule comprises a nucleotide sequence encoding the human pheromone receptor, hV3R8. In one embodiment, the nucleic acid molecule comprises the sequence as set forth in SEQ ID NO: 3. More specifically, the nucleic acid molecule may be a DNA or RNA derived from human tissue, cells or cell lines or a recombinant DNA. Still more specifically, the nucleic acid molecule may be a genomic DNA, cDNA, or mRNA.

In another aspect there is provided an isolated nucleic acid molecule that hybridizes under high, medium or low stringency conditions to a nucleic acid encoding a polypeptide of SEQ ID NO: 4, wherein the polypeptide mediates a physiological and/or behavioral effect in mammals. Preferably, the polypeptide mediates a physiological and/or behavioral effect in humans. In a preferred embodiment, the isolated nucleic acid molecule is a DNA or RNA oligonucleotide probe, wherein the oligonucleotide probe is about 7 to about 50 nucleotides in length and, optionally, comprises a detectable label.

In another aspect there is provided a vector comprising a nucleotide sequence encoding a polypeptide of SEQ ID NO: 4, or polypeptide fragment thereof, wherein the polypeptide or polypeptide fragment mediates a physiological and/or behavioral effect in mammals. The polypeptide may be expressed in mammalian-, bacterial-, insect- or yeast cells that are stably transduced or transiently transfected with the vector. In a preferred embodiment, the polypeptide is expressed as a cell-surface receptor, including a functional or partially functional receptor.

In another aspect there is provided an isolated polypeptide of SEQ ID NO: 4, and analogs and fragments of the isolated polypeptide, wherein the polypeptide or polypeptide fragment mediates a physiological and/or behavioral effect in mammals. More particularly, the amino acid sequence of the isolated polypeptide comprises SEQ ID NO: 4.

In a further aspect of the invention there is provided polyclonal or monoclonal antibodies, and fragments thereof, that specifically bind to an isolated polypeptide of SEQ ID NO: 2 or fragments thereof, wherein the polypeptide mediates a physiological and/or behavioral effect in mammals. In a preferred embodiment, the monoclonal antibodies are fully or partially humanized antibodies or chimeric antibodies.

In another aspect there are provided agents that specifically bind to an isolated polypeptide of SEQ ID NO: 2 or fragments thereof, wherein the polypeptide mediates a physiological and/or behavioral effect in mammals. The agents include, but are not limited to, pheromones. More specifically, the agents may be agonists or antagonists of the hV3R1 receptor. Furthermore, the agents may be isolated from natural sources, prepared by synthetic methods, or derived from the synthetic transformation of isolated natural sources.

In a further aspect, there is provided a composition comprising an isolated nucleic acid molecule, isolated polypeptide, isolated polyclonal or monoclonal antibody, or agent of the present invention. For example, the composition may comprise a pheromone. The nucleic acid molecule is a DNA or RNA comprising a nucleotide sequence encoding a polypeptide of SEQ ID NO: 2 or fragment thereof; or a nucleotide sequence that hybridizes or is complementary to the nucleotide sequence encoding a polypeptide of SEQ ID NO: 2 or fragment thereof, wherein the polypeptide mediates a physiological and/or behavioral effect in mammals. More particularly, the nucleic acid molecule comprises a DNA or RNA encoding a human pheromone receptor.

In a further aspect of the invention there is provided polyclonal or monoclonal antibodies, and fragments thereof, that specifically bind to an isolated polypeptide of SEQ ID NO: 4 or fragments thereof, wherein the polypeptide mediates a physiological and/or behavioral effect in mammals. In a preferred embodiment, the monoclonal antibodies are fully or partially humanized antibodies or chimeric antibodies.

In another aspect there are provided agents that specifically bind to an isolated polypeptide of SEQ ID NO: 4 or fragments thereof, wherein the polypeptide mediates a physiological and/or behavioral effect in mammals. The agents include, but are not limited to, pheromones. More specifically, the agents may be agonists or antagonists of the hV3R8 receptor. Furthermore, the agents may be isolated from natural sources, prepared by synthetic methods, or derived from the synthetic transformation of isolated natural sources.

In a further aspect, there is provided a composition comprising an isolated nucleic acid molecule, isolated polypeptide, isolated polyclonal or monoclonal antibody, or agent of the present invention. For example, the composition may comprise a pheromone. The nucleic acid molecule is a DNA or RNA comprising a nucleotide sequence encoding a polypeptide of SEQ ID NO: 4 or fragment thereof; or a nucleotide sequence that hybridizes or is complementary to the nucleotide sequence encoding a polypeptide of SEQ ID NO: 4 or fragment thereof, wherein the polypeptide mediates a physiological and/or behavioral effect in mammals. More particularly, the nucleic acid molecule comprises a DNA or RNA encoding a human pheromone receptor.

In another aspect of the invention, there is provided a composition comprising a fragment of the isolated nucleic acid, polypeptide, or polyclonal or monoclonal antibody of the present invention.

Another aspect of the invention provides a method for mediating a physiological disorder comprising administering to a subject an effective amount of a composition comprising an isolated nucleic acid molecule encoding a polypeptide of SEQ ID NO: 2 or SEQ ID NO: 4, or polypeptide fragment(s) thereof, and inducing expression thereof, wherein the subject is in need of treatment for diseases or disorders including, but not limited to, infertility; disorders related to contraception; disorders related to hormonal regulation, sexual dysfunction, and erectile dysfunction; neurological-, psychiatric-, liver- and spleen disorders; cardiovascular disorders; cancer; and known or unknown disorders related to the aberrant expression of the human pheromone receptor hV3R1 or hV3R8.

Yet, another aspect of the invention provides a method for mediating a physiological disorder comprising administering an effective amount of an isolated polypeptide comprising the amino acid sequence set forth in SEQ ID NO: 2 or SEQ ID NO: 4, or fragment(s) thereof, to a subject, wherein the subject is in need of treatment for diseases or disorders selected from the group consisting of infertility; disorders related to contraception; disorders related to hormonal regulation, sexual dysfunction, and erectile dysfunction; neurological-, psychiatric-, liver- and spleen disorders; cardiovascular disorders; cancer; and known or unknown disorders related to the aberrant expression of the human pheromone receptor hV3R1 or hV3R8.

Still another aspect of the invention provides a method for mediating a physiological disorder comprising administering a composition which includes an isolated polyclonal or monoclonal antibody that specifically binds to an isolated polypeptide comprising the amino acid sequence set forth in SEQ ID NO: 2 or SEQ ID NO: 4, or fragment(s) thereof, wherein the subject is in need of treatment for diseases or disorders. Examples of such diseases or disorders include, but are not limited to, infertility; disorders related to contraception; disorders related to hormonal regulation, sexual dysfunction, and erectile dysfunction; neurological-, psychiatric-, liver- and spleen disorders; cardiovascular disorders; cancer; and known or unknown disorders related to the aberrant expression of the human pheromone receptor hV3R1 or hV3R8. The method for mediating a physiological disorder includes administering to the subject an effective amount of the composition by topical, oral, inhalable, subcutaneous or intramuscular administration.

The invention also encompasses a method for detecting a target nucleic acid molecule in a sample using a probe that comprises a nucleotide sequence that hybridizes or is complementary to a sequence of the nucleotide sequence of the target nucleic acid molecule. The target nucleic acid molecule encodes a polypeptide of SEQ ID NO: 2, SEQ ID NO: 4, or fragment(s) thereof, wherein the polypeptide mediates a physiological and/or behavioral effect in mammals. The method includes exposing the probe to a sample, under conditions where the probe hybridizes under high, medium or low stringency conditions to the target nucleic acid molecule in the sample, and detecting the target nucleic acid molecule hybridized to the probe. In a preferred embodiment, the probe is about 7 to about 50 nucleotides in length. In another preferred embodiment, the probe has a detectable label. In another preferred embodiment, the probe is immobilized or in solution. In yet another preferred embodiment, the probe is selected from the group consisting of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, fragment(s) of SEQ ID NO: 1, and fragment(s) of SEQ ID NO: 3.

In another aspect of the invention, there is provided a method for identifying an agent that specifically binds to an isolated polypeptide of the present invention. The method employs a polypeptide of SEQ ID NO: 2, SEQ ID NO: 4, or fragment(s) thereof, wherein the polypeptide mediates a physiological and/or behavioral effect in mammals. The method includes exposing the polypeptide to a sample, under conditions where the agent in the sample specifically binds to the polypeptide; and detecting the agent specifically bound to the polypeptide.

In another aspect, there is provided a transgenic animal that expresses the polypeptide encoded by the amino acid sequence of SEQ ID NO: 2 or SEQ ID NO: 4. In a preferred embodiment, the transgenic animal is a rodent, e.g., a mouse or rat.

In another aspect, there is provided a diagnostic assay that determines if a subject will benefit from the modulation of the expression, function, or activity of a human pheromone receptor; or from gene therapy using a vector that is capable of expressing the human pheromone receptor. Particularly, there is provided a genetic test that determines if a subject will benefit from the modulation of the expression, function, or activity of a human pheromone receptor; or from gene therapy using a vector that is capable of expressing the human pheromone receptor.

In a further aspect, there is provided a kit for detecting a nucleic acid molecule of SEQ ID NO: 1, SEQ ID NO: 3, or fragment(s) thereof, using as a probe an isolated nucleic acid molecule that hybridizes to or has a nucleotide sequence complementary to the sequence of the nucleic acid molecule encoding a polypeptide of the instant invention.

Also contemplated by the present invention are antisense oligonucleotides capable of hybridizing to a target nucleic acid molecule encoding a human pheromone receptor and, thereby, modulating the expression, function or activity of the human pheromone receptor. The antisense oligonucleotide is an isolated, modified or synthetic nucleic acid molecule, and may be a DNA or RNA. Likewise, the target nucleic acid molecule may be a DNA or RNA. The length of the antisense oligonucleotides may depend on a number of factors, including for example, the sequence of the target nucleic acid molecule, and the desired specificity of binding to the target nucleic acid molecule. Antisense oligonucleotides that may be effective in modulating, for example inhibiting or reducing, the expression of a nucleic acid molecule encoding a human pheromone receptor are preferably at least 7 nucleotides in length, more preferably 15 nucleotides in length and most preferably 20-30 nucleotides in length. However, the antisense oligonucleotides may be any length appropriate for use in modulating expression of the target nucleic acid molecule. Also, modified oligonucleotides of only 7 bases can be effective antisense oligonucleotides (see Wagner et al. (1996) Nature Biotechnology 14:840-844). Antisense oligonucleotides may contain, for example, bases with standard 5′-3′ phosphodiester linkages or may contain modified bases (e.g., methylated bases), modified sugars (e.g., methylated sugars), non-ribose sugars, or various alternative linkages (e.g., phosphorothioate, carbamate, peptide, alkylphosphonate, phosphoroamidate, acetamidate, etc.), and/or mixtures of normal/modified bases and standard/altemative linkages.

In another aspect, an expression vector is contemplated that, when introduced into cells by techniques well known in the art, directs the synthesis of an antisense RNA that hybridizes to complementary sequences in the cells and modulates, i.e., inhibits or reduces expression of the receptor, in vitro or in vivo.

In yet another aspect, there is provided a kit for detecting a polypeptide having an amino acid sequence of SEQ ID NO: 2, SEQ ID NO: 4, or fragment(s) thereof, using as a probe the polyclonal or monoclonal antibody of the present invention.

c) Identification, Cloning and Characterization of Human Pheromone Receptors

The novel vomeronasal organ (VNO) receptors hV3R1 and hV3R8 are identified via a Bioinformatics approach. A human sequence database is searched for potential open reading frames (ORF) capable of encoding a polypeptide that has homology or is related to a putative or known pheromone receptor. PCR primers can later be synthesized and used to amplify the nucleic acid molecule containing the nucleotide sequence(s) identified from the sequence database search (target nucleic acid molecule). For example, the GenBank database for High Throughput Genomic (HTG) Sequences and/or the Human Genome Database (HGD) at the National Center for Biotechnology Information (NCBI), as well as the ENSEMBL database at the European Biotechnology Institute (EBI), and/or other Genomic databases may be searched for an open reading frame capable of encoding a human homologue of a putative or known mouse pheromone receptor (vide infra).

A query set may be built by keyword search on the National Center for Biotechnology Information (NCBI) and the Olfactory Receptor Database at Yale University. BLAST searches can be performed with full-length cDNA and amino acid sequences of putative human VNO receptors that are available in the public domain, e.g., V1RL1 (Rodriguez et al.), FKSG 46, 47 and 83 (Wang). When amino acid sequences are used as probes, baits, and/or templates, TBLASTN algorithm can be employed. Different databases may be employed (supra). In addition to the human receptor sequences, representative mouse/rat sequences may also be used in BLAST searches in the databases. The resulting output from all of the searches is sorted out to identify open reading frames (ORFs) that are contiguous with or without frameshifts. A large number of pseudogenes may be identified in the search output. Furthermore, a sequence may be deemed a potential pseudogene by the presence of one or more stop codons in a reading frame in the coding region and by the absence of a contiguous coding region in the other two reading frames.

The genomic sequences with ORFs can be exported for translation via specific software (e.g., GenTool, Double Twist, Inc.) in order to obtain the putative receptor sequences. In addition, searches can be performed with the DNA sequences using algorithms available through the software in order to gain further information about the homology of the novel sequences, including expressed sequence tags (ESTs). The translated protein sequences are analyzed for membrane spanning domains and other characteristic features present in VNO receptors and other members of the super gene family of G-protein coupled receptors (GPCR).

PCR primers may be designed to amplify the target nucleic acid molecule, encoding a polypeptide with homology to a putative or known receptor, from DNA or RNA prepared from human tissue, cells or cell lines. For example, genomic DNA or cDNA prepared from human tissue, cells or cell lines may be used as a template for the PCR. The cDNA may be synthesized from mRNA prepared from human tissue, cells or cell lines, and amplified using RT-PCR. Further, a collection of cDNAs representative of the population of mRNAs in a cell or tissue, i.e., a cDNA library may be prepared and used as a template for the PCRs. In this manner, a randomly primed human VNO cDNA library may be prepared. Mixed hexamers can randomly prime first-strand cDNA synthesis along the poly(A)⁺ human VNO mRNA; the reactions can be incubated at about 45° C. to melt potential secondary structures in the template mRNA. Second strands can be synthesized using E. coli DNA polymerase I in combination with RNase H and DNA ligase. In the final step, T4 DNA polymerase may fill in and blunt the ends of the randomly primed double-stranded cDNA. The cDNA may be ligated to an excess of commercially available adaptor, for example, Eco RI (Not, Sal) adapter. The adapter usually contains the recognition sites for restriction enzymes such as Not I and Sal I to facilitate subsequent excision of the insert from the vector. However, the restriction enzymes are likely to cut the cDNA inserts only infrequently, if at all. The randomly primed double-stranded cDNA may then be non-directionally cloned into a suitable vector that has been linearized with, for example, Eco RI, and treated with phosphatase. The ligated DNA may be transfected into competent E. coli (e.g., DH1OB, TOP10). The randomly primed library can then be screened at high stringency, using a probe derived from the 5′ end of individual human receptor cDNAs, to identify overlapping fragments that can be assembled into a full-length cDNA clone.

It is readily apparent to those skilled in the art that DNA encoding such a polypeptide may also be amplified from a genomic DNA library. Construction of genomic libraries can be performed by standard methods well known in the art and can be found in Maniatis T., Fritsch, E. F., Sambrook, J. in Molecular Cloning: A Laboratory Manual (Cold Spring Harbor Laboratory, Cold Spring Harbor, N.Y., 1989).

The PCR of a target nucleic acid molecule, for example a genomic DNA or cDNA, may be primed using pairs of oligonucleotide primers that are synthesized and have a sequence that is complementary to a sequence in the sense or antisense sequence of the target nucleic acid molecule. The sequence of the oligonucleotides may be based on the sequence of the target nucleic acid molecule identified from the sequence database search.

The amplified target nucleic acid molecule may then be cloned, sequenced and expressed, and the encoded and expressed polypeptide may be characterized using standard techniques. Techniques for identifying, cloning, sequencing, expressing and characterizing nucleic acid and/or polypeptides are well known to those skilled in the art. Also, PCR, RT-PCR, nucleic acid hybridization and other techniques used for screening of nucleic acids are well known to those of skill in the art and the selection of such techniques does not limit the present invention. Further, the procedures for isolating and identifying fragments of a nucleic acid molecule or polypeptide are well known to those of skill in the art; see, e.g., T. Maniatis et al., Molecular Cloning: A Laboratory Manual, Cold Spring Harbor Laboratory Press.

The sequence of the amplified nucleic acid molecule and the encoded polypeptide may be compared to the nucleotide sequence of the target nucleic acid molecule identified from the sequence data base search and/or corresponding homologous or related sequences, and thereby a sequence encoding a homologous or related human pheromone receptor may be identified. Moreover, differences between the newly identified sequences and corresponding homologous or related sequences may be identified. For example, the sequence of a receptor, encoded by the amplified target nucleic acid molecule may be aligned with the corresponding homologous or related sequences of known or putative human or rodent VNO receptors, particularly pheromone receptors, to determine specific sequence and structure differences and percent homology or relatedness between the sequences. Using this approach, novel human pheromone receptors, may be identified, cloned and characterized.

d) Preparation and Use of Nucleic Acid Probes

The nucleic acid molecule, for example DNA or RNA, encoding a human pheromone receptor, or fragments thereof, may be readily synthesized by methods well known in the art, for example, by solid phase oligonucleotide synthesis (Letsinger et al., (1965) Oligonucleotide synthesis on a polymer support. J. Am. Chem. Soc. 87:3526-3227). Alternatively, for example, the DNA or RNA encoding the receptor, or a fragment thereof, may be produced by recombinant methods. Such methods are described by T. Maniatis et al, Molecular Cloning (A Laboratory Manual), Cold Spring Harbor Laboratory (1982). Further, nucleic acid hybridization or amplification methods can be performed using known probes.

Probes may be cloned and prepared from a nucleic acid molecule, for example DNA or RNA, encoding a human pheromone receptor, or a fragment thereof. Thus, any recombinant nucleic acid molecule containing such DNA or RNA, or fragment thereof, is contemplated herein. The cloned DNA or RNA, or fragment thereof, according to the invention may be used as a nucleic acid hybridization probe for detection of identical, homologous or related nucleotide sequences. Further, the probe may contain a detectable label. Methods for preparing such probes are well known in the art.

Also contemplated herein are oligonucleotide probes containing a sequence derived from the human pheromone receptor hV3R1 and hV3R8 (e.g., SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, fragment(s) of SEQ ID NO: 1, and fragment(s) of SEQ ID NO: 3). Such oligonucleotide probes may contain a sequence complementary to a sequence in a DNA or RNA sequence encoding a human pheromone receptor. Under high stringency nucleic acid hybridization conditions, the oligonucleotide probes may detect a nucleotide sequence of a nucleic acid molecule, for example a DNA or RNA, encoding the human pheromone receptor, or under reduced nucleic acid stringency hybridization conditions the oligonucleotide probes may detect sequences that are homologous or related to the sequence of the nucleic acid molecule encoding a human pheromone receptor. As used herein, homologous or related sequences may share a level of homology with a sequence, particularly the sequence of a known or putative pheromone receptor. For example, a sequence may be at least 40% homologous to a sequence of a known or putative pheromone receptor. In other examples, the level of homology may be 45%, 50%, 75%, or a higher percent homology. In a preferred embodiment, the probe is about 7 to 50 nucleotides in length. In another preferred embodiment, the probe has a detectable label. In another preferred embodiment, the probe is immobilized or in solution.

Such methods and probes can be used for diagnostic purposes to: determine whether a nucleic acid molecule, for example a DNA or RNA, encoding a particular human pheromone receptor is present in the VNO and/or other tissues of a patient; determine whether or not a particular human pheromone receptor is expressed specifically or differentially in males and in females, or in a particular species, cell or tissue type; detect differences in the nucleotide or amino acid sequences encoding human pheromone receptors; detect the level of expression between alleles of a particular human pheromone receptor; and distinguish between functional and nonfunctional or mutant alleles of a particular human pheromone receptor. The sequences distinguishing between functional and nonfunctional or mutant alleles of a particular human pheromone receptor may comprise a point mutation and sequences flanking or extending from the point mutation, as in single nucleotide polymorphisms.

For example, a method for screening for functional or non-functional or mutant alleles of the human pheromone receptor may comprise the following steps: extraction of DNA from tissue, restriction enzyme cleavage of the extracted DNA, electrophoresis of the cleaved DNA fragments and detection of single-base mutations in the DNA using, for example, mismatch repair enzymes (Lu et al., Genomics 14:249-55 (1992), or screening for mutations by enzyme mismatch cleavage with T4 endonuclease VII (Youil et al., Proc. Natl. Acad. Sci. USA 92:87-91 (1995). In situ hybridization methods may also be used.

e) Preparation and Use of Nucleic Acid Molecules and Polypeptides

The nucleic acid molecule encoding a human pheromone receptor may be inserted into a vector and used for replication of the nucleic acid molecule and/or expression of the cloned human receptor, or fragment thereof. A vector construct for replicating the nucleic acid molecule or expressing the receptor may comprise, for example: (i) a sequence encoding a human pheromone receptor, or fragment thereof, and (ii) control or regulatory sequences operably linked to the sequence encoding the receptor, or fragment thereof, so that the receptor may be expressed in host cells. The control or regulatory sequences effecting expression of the receptor in host cells may comprise, for example, promoter and/or enhancer sequences. The control or regulatory sequences may further be specific to a particular cell or tissue type and/or comprise a negative regulatory element that is activated when the host cells undergo differentiation. Further, the host cells may be cells competent for use in the production of transgenic animals.

Expression constructs may be designed to express antisense DNA or RNA in vitro or in vivo. The expression construct may comprise, for example, a sequence encoding an antisense DNA or RNA capable of inhibiting the expression of a human pheromone receptor. The sequence of the antisense DNA or RNA may be based on a sequence encoding a human pheromone receptor and may be complementary to a sequence in the DNA or RNA sequence encoding the receptor.

The nucleic acid molecules and the polypeptides produced from such vectors are useful in the compositions and methods of the present invention. For example, the expressed receptor polypeptides may be used as a probe to detect agents that specifically bind to the receptor. Such polypeptide probes may optionally contain a detectable label and may be prepared by methods well known in the art.

f) Preparation and Use of Transduced or Transfected Cells

Cells may be transduced or transfected with DNA encoding a particular polypeptide, and the encoded polypeptide transiently or stably expressed by the transfected or transduced cells. For example, an expression construct comprising DNA encoding a human pheromone receptor, or fragment thereof, may be introduced into cells by methods well known in the art, such as calcium phosphate precipitation; lipid-based or viral mediated methods; or electroporation. To select positively transduced or transfected cells containing the expression construct, the cells may be cultured for 1-2 days in enriched medium and then switched to a selection medium. The selectable marker of the expression construct may confer resistance to the selection medium and allow the positively transduced or transfected cells to grow and form foci which in turn can be cloned and expanded into cell lines. The resulting cell lines may express the desired human pheromone receptor, or fragment thereof, on the surface of the cell and, thus, are particularly useful for screening candidate drugs that specifically bind to the receptor, or fragment thereof. Thus, the resulting cell lines may be used to develop automated high throughput screening assays to identify novel compounds, e.g., agents or ligands, of the expressed polypeptide with therapeutic utility in the treatment of certain disorders and diseases including, but not limited to, infertility, disorders related to contraception, disorders related to hormonal regulation, sexual dysfunction, erectile dysfunction; neurological-, psychiatric-, liver- and spleen disorders; cardiovascular disorders; cancer; and known or unknown disorders related to the aberrant expression of pheromone receptor(s) or mediated by pheromone receptor(s).

Cells capable of expressing a human pheromone receptor include, but are not limited to, mammalian-, bacterial-, insect- and yeast cells. Selection of suitable cells or cell lines may be determined, for example, by Southern or northern blot analysis, radio-ligand binding analysis, functional analysis, or by screening for the receptor on the cell surface (e.g., FACS). Methods for detecting the receptor or receptor activity are well known in the art. For example, receptor expression or activity in a cell can be detected or measured by the binding of radiolabeled ligand to the receptor. The cells expressing the receptor or having receptor activity may be suitable for isolation of the DNA or RNA encoding the receptor.

g) Preparation and Use of Antibodies

Antibodies directed against a particular human pheromone receptor, or fragment thereof, may be produced by methods well known in the art and may be useful, for example, in the diagnosis and treatment of diseases and disorders associated with the overexpression of the receptor, and to assess the state of physiological functioning of the receptor.

Polyclonal antibodies directed against a particular polypeptide, may be prepared and produced using well-established techniques involving immunization of an animal, for example, a rabbit, guinea pig, or goat, with an appropriate immunogen, for example, a preparation containing a polypeptide of interest.

Monoclonal antibodies may be prepared and produced using well-established techniques, for example, using somatic cell hybridization techniques or the standard techniques of Kohler and Milstein, (Nature 265:495-497, 1975). Moreover, humanized or chimeric antibodies may be prepared, isolated and produced by methods well known in the art, including antibody phage display libraries. For reviews on techniques for preparing and producing monoclonal antibodies, see Birch et al., (1995) Monoclonal Antibodies: principles and application, Wiley-Liss, N.Y.; Davis (1995) Monoclonal antibody protocols, Humana Press, Totowa, N.J., and for preparing humanized or chimeric antibodies see Merluzzi et al., (2000) Humanized antibodies as potential drugs for therapeutic use, Advanced Clinical Pathology 4 (2):77-85, and Kipriyanov et al., (1999) 12 (2):173-201.

In order to immunize an animal, for example a mouse, samples of an appropriate immunogen preparation may be injected into the animal. After a sufficient time for antibodies to be produced by the immunized animal, the animal is sacrificed and spleen cells obtained. Alternatively, the spleen cells of a non-immunized animal may be sensitized to the immunogen in vitro. The spleen cell chromosomes encoding the desired immunoglobins can then be expressed by fusing the spleen cells, generally in the presence of a non-ionic detergent, for example, polyethylene glycol, with a myeloma cell line. The resulting cells, which include fused hybridomas, are allowed to grow in a selective medium, such as medium containing hypoxanthine, aminopterin and thymidine (HAT medium). The surviving immortalized cells are then cultured in HAT medium under limiting dilution conditions in a suitable container, for example microtiter wells, and the supernatant screened for monoclonal antibodies having the desired specificity.

The yield of monoclonal antibodies may be increased by, for example, injecting hybridoma cells into the peritoneal cavity of a mammalian host that accepts the cells so that the antibodies can be produced in the animal, and subsequently harvesting the ascites fluid of the animal containing the monoclonal antibodies. Where an insufficient amount of the monoclonal antibody collects in the ascites fluid, the antibody may be harvested from the blood of the host. Alternatively, the cells producing the desired antibody can be cultured and expanded, for example, in a hollow fiber cell culture device or a spinner flask device, or using other techniques well known in the art. Further, techniques for the isolation and purification of monoclonal antibodies are well-known in the art (see Kohler and Milstein, supra). For example, the sequence coding for an antibody binding site can be excised from chromosomal DNA, inserted into a cloning vector and the encoded antibody binding site expressed in bacteria to produce recombinant polypeptides having the corresponding antibody binding sites.

In general, antibodies can be purified by known chromatographic techniques, for example, Polypeptide A chromatography, Polypeptide G chromatography, DEAE chromatography, ABx chromatography, and filtration chromatography. Antibodies may be used as diagnostic tools such as to determine if an individual expresses a specific receptor, particularly a specific pheromone receptor. This information will be useful for determining whether a compound or drug, for example a pheromone, that binds to the encoded receptor will modulate a physiological, behavioral, and/or therapeutic response in the individual. Other uses include delivering or administering the antibodies as a therapeutic to: 1) inhibit or reduce binding of a compound, for example a pheromone, to a pheromone receptor, and more particularly to a pheromone receptor displayed or expressed on the cell surface; and/or 2) to prevent the pheromone receptor from interacting with and/or transducing a signal to its binding partners. Further, the antibodies may be used as a probe for detecting the receptor and may optionally contain a detectable label.

h) Screening for Agents that Modulate Receptor Activity

The present invention is also directed to methods for screening for agents that modulate in vivo the expression, function or activity of a pheromone receptor and/or the nucleic acid molecule encoding a pheromone receptor. Examples of such activity include binding activity and/or signal transduction activity of the receptor, and/or physiological or behavioral effects mediated by the receptor. Agents that modulate such expression, function or activity of a pheromone receptor may be detected by a variety of assays well known in the art, including high-throughput screening assays. For example, an assay procedure to identify an agent that specifically binds or interacts with a pheromone receptor may contain the receptor of the present invention, and a candidate agent or test sample that contains a putative agent. The candidate agent or test sample may be tested directly on, for example, purified native or recombinant receptor polypeptide, subcellular fractions of receptor-producing native or recombinant cells and/or whole cells expressing the native or recombinant receptor. Modulators identified using such assays are useful as therapeutic and diagnostic agents. The candidate agent or test sample may be added to the receptor in the presence or absence of a known receptor ligand with or without a detectable label.

The modulating effect of the candidate agent or test sample may be determined, for example, by analyzing the ability of the candidate agent or test sample to bind to the receptor; modulate the binding of other compounds (or other agents) to the receptor; and/or modulate a physiological or behavioral effect mediated by the receptor. In general, an assay for identifying modulators will contain a receptor of the present invention, and a candidate agent or test sample that contains a putative modulator. The candidate agents or test samples may be tested directly, for example, on purified native or recombinant receptor, subcellular fractions of cells producing native or recombinant receptor, and/or whole cells expressing native or recombinant receptor. The candidate agent or test sample may be added to the receptor in the presence or absence of a known receptor ligand optionally containing a detectable label.

The identification and isolation of modulators of pheromone receptor activity are useful in treating disorders mediated by the receptor. Other agents may be useful for stimulating or inhibiting activity of the receptor. Selective agonists or antagonists of the receptor may be used to treat disorders and diseases including, but not limited to, infertility, disorders related to contraception, disorders related to hormonal regulation, sexual dysfunction, erectile dysfunction; neurological-, psychiatric-, liver- and spleen disorders; cardiovascular disorders; cancer; and known or unknown disorders related to the aberrant expression of pheromone receptor(s) or mediated by pheromone receptors. Modulators include, for example, the antibodies of the present invention.

i) Preparation, Use and Delivery of Antisense Oligonucleotides

Also contemplated by the present invention are antisense oligonucleotides capable of hybridizing to a target nucleic acid molecule encoding a human pheromone receptor and, thereby, modulating the expression, function or activity of the receptor, for example a human pheromone receptor. The antisense oligonucleotide is an isolated, modified or synthetic nucleic acid molecule, and may be a DNA or RNA. Likewise, the target nucleic acid molecule may be a DNA or RNA. The length of the antisense oligonucleotides may depend on a number of factors, including for example, the sequence of the target nucleic acid molecule, and the desired specificity of binding to the target nucleic acid molecule. Antisense oligonucleotides that may be effective in modulating, for example inhibiting or reducing, the expression of a nucleic acid molecule encoding a human pheromone receptor are preferably at least 7 nucleotides in length, more preferably 15 nucleotides in length and most preferably 20-30 nucleotides in length. However, the antisense oligonucleotides may be any length appropriate for use in modulating expression of the target nucleic acid molecule. Also, modified oligonucleotides of only 7 bases can be effective antisense oligonucleotides (see Wagner et al. (1996) Nature Biotechnology 14:840-844). Antisense oligonucleotides may contain, for example, bases with standard 5′-3′ phosphodiester linkages or may contain modified bases (e.g., methylated bases), modified sugars (e.g., methylated sugars), non-ribose sugars, or various alternative linkages (e.g., phosphorothioate, carbamate, peptide, alkylphosphonate, phosphoroamidate, acetamidate, etc.), and/or mixtures of normal/modified bases and standard/alternative linkages. In another approach, an expression vector is contemplated that, when introduced into cells by techniques well known in the art, directs the synthesis of an antisense RNA that hybridized to complementary sequences in the cells and modulates, for example inhibits or reduces expression of the receptor, in vitro or in vivo.

j) Preparation, Use and Delivery of Compositions

Pharmaceutically useful compositions of the present invention may be formulated according to known methods such as by the admixture of a pharmaceutically acceptable carrier. Examples of such carriers and methods of formulation may be found in Remington: The Science and Practice of Pharmacy, A. Gennaro, ed., 20th edition, Lippincott, Williams & Wilkins, Philadelphia, Pa. To form a pharmaceutically acceptable composition suitable for effective administration, such compositions will contain an effective amount of the nucleic acid molecule, polypeptide, agent or other product of the present invention.

Therapeutic or diagnostic compositions of the present invention are administered to a subject in amounts sufficient to treat or diagnose disorders in which modulation of the activity mediated by the human pheromone receptor is indicated. The effective amount may vary according to a variety of factors such as the individual's condition, weight, sex and age. Other factors include the mode of administration. The pharmaceutical compositions may be provided or delivered to the subject by a variety of routes such as topical, oral, subcutaneous, and intramuscular. Compositions may take the form of solutions, suspensions, aerosols, or any other appropriate compositions; and comprise at least one compound of this invention in combination with at least one pharmaceutically acceptable excipient. Suitable excipients are well known to persons of ordinary skill in the art, and the methods of formulating the compositions, may be found in such standard references as “Remington: The Science and Practice ofPharmacy”. The amount of a compound of this invention in the composition may vary widely depending on the type of composition, size of a unit dosage, kind of excipients, and other factors well known to those of ordinary skill in the art. In general, the final composition may comprise from 0.000001 percent by weight (% w) to 10% w of the compound of this invention, preferably 0.00001% w to 1% w, with the remainder being the excipient or excipients.

k) Methods and Use for Gene Therapy

Nucleic acid molecules containing nucleotide sequences that are complementary to a sequence of the nucleotide sequence encoding a human pheromone receptor can be designed and synthesized for use in antisense therapy. For example, the antisense oligonucleotides may be the stable derivatives of DNA such as phosphorothioates or methylphosphonates, or of RNA such as 2′-O-alkyl-RNA, or other antisense oligonucleotide mimetics. Such antisense oligonucleotides may be introduced into cells by methods known in the art, including microinjection, liposome encapsulation or by expression from vectors containing the antisense nucleotide sequence. Antisense therapy may be particularly useful for the treatment of disorders where it is beneficial to inhibit or reduce the activity of a pheromone receptor. In particular, modulating the expression of a pheromone receptor may be desirable in the treatment of certain disorders and diseases including, but not limited to, infertility, disorders related to contraception, disorders related to hormonal regulation, sexual dysfunction, erectile dysfunction; neurological-, psychiatric-, liver- and spleen disorders; cardiovascular disorders; cancer; and known or unknown disorders related to the aberrant expression and/or over-expression of a specific pheromone receptor.

Nucleic acid molecules encoding a human pheromone receptor may be used to introduce the receptor into the cells of a target organism for gene therapy. Such gene therapy is also described herein as “receptor gene therapy.” The target organism may be any subject in need of treatment and is an animal, for example, a human or a rodent. The nucleic acid molecule encoding the receptor may be ligated into, for example, viral vectors which mediate transfer of the nucleic acid molecule by infection of recipient host cells. Suitable viral vectors include, for example, retrovirus, adenovirus, adeno-associated virus, herpes virus, vaccinia virus and polio virus. Alternatively, the nucleic acid molecule encoding a human pheromone receptor can be transferred into cells for gene therapy using non-viral techniques, including receptor-mediated targeted DNA transfer using ligand-DNA conjugates or adenovirus-ligand-DNA conjugates, lipofection membrane fusion or direct microinjection. These procedures and variations thereof are suitable for ex vivo as well as in vivo receptor gene therapy. Tolstoshev, P. Gene therapy, concepts, current trials and future directions. Annu. Rev. Pharmacol. Toxicol. (1993) 33:573-96; Wilson, J. M. Vectors--shuttle vehicles for gene therapy. Clin. Exp. Immunol. (1997) 107 Suppl. 1:31-32; Karson, E. M. Prospects for gene therapy. Biol. Reprod. (1990) 42:39-49.

I) Preparation and Use of Kits

Kits containing the isolated and synthetic nucleic acid molecules, polypeptides and agents of the present invention, for example the DNA, RNA, recombinant polypeptides and antibodies of the present invention, may be prepared. Such kits may be used, for example, to detect a nucleic acid molecule encoding a pheromone receptor to detect the presence of, and/or quantify, the receptor polypeptide, or fragments thereof, in a sample. Such characterization is useful for a variety of purposes, including but not limited to forensic analysis, monitoring the course of therapy and epidemiological studies.

The isolated and synthetic nucleic acid molecules, polypeptides and agents of the present invention, for example the DNA, RNA, recombinant polypeptides and antibodies of the present invention, may be used to detect, screen or measure levels of DNA or RNA encoding the receptor or receptor polypeptide. Such DNA, RNA, recombinant polypeptides, antibodies and agents lend themselves to the formulation of kits suitable for the detection, quantification, and typing of pheromone receptors. Such a kit would comprise, for example, a carrier suitable for holding in close confinement at least one container. The carrier would further comprise reagents, for example, the isolated or synthetic nucleic acid molecules, polypeptides and agents of the present invention.

m) Preparation and Use of Transgenic Animals

The transduced or transfected cells may provide the basis for the development of transgenic animals. For example, transfected germline-competent cells may be introduced into an early stage embryo, such as a normal blastocyst. Transgenic animals are preferably a mammal, more preferably a rodent and most preferably a mouse. The methods for producing a transgenic animal are well known in the art. For example, a transgenic animal may be developed by introducing into embryonic cells an expression construct comprising a sequence encoding a human pheromone receptor, or fragment thereof, operably linked to control or regulatory sequences sufficient for effecting expression of the encoded polypeptide in germline-competent cells. The control or regulatory sequences of the expression construct may comprise at least one sequence that is specifically or exclusively expressed in cells that are germline-competent. The cells containing the expression construct may then be transferred to a recipient embryo, where the genome of the recipient embryo differs from that of the transferred cells. In a preferred embodiment, at least some of the transferred cells contribute to the development of the embryo. The recipient embryo is then allowed to develop at least for the full gestational period of the animal. Ledermann B., Embryonic stem cells and gene targeting. Exp. Physiol. (2000) 85:603-13; Maroulakou, I. G. and Muise-Helmericks, R. C., Guide to techniques in creating transgenic mouse models using SV40 T antigen. Methods Mol. Biol. (2001) 165:269-86.

n) Examples

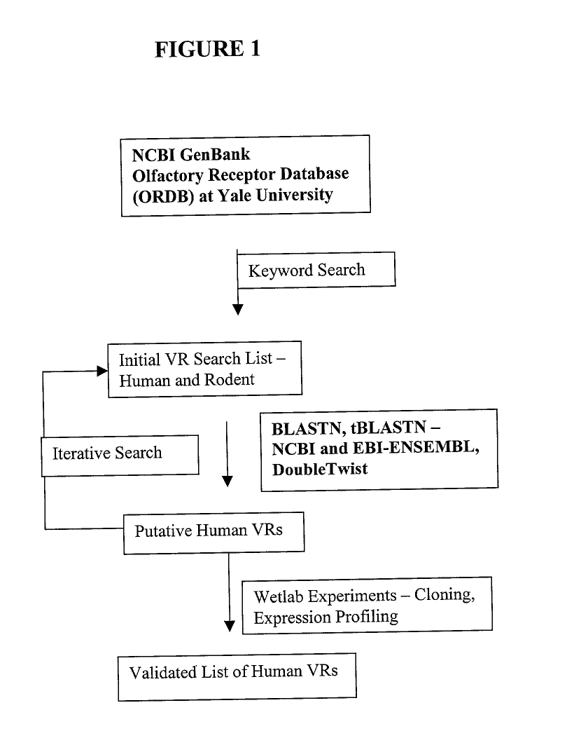

The following specific examples are intended to illustrate the invention and should not be construed as limiting the scope of the claims. The examples further illustrate some of the specifics of identifying novel vomeronasal organ (VNO) receptors and methods employed. A schema for the workflow of performed experiments is shown in FIG. 1.

I. Bioinformatics Methods used for the Identification of the Novel Vomeronasal Organ (VNO) Receptors

The novel vomeronasal organ (VNO) receptors hV3R1 and hV3R8 were first identified via a Bioinformatics approach. A query set was built by keyword search on the National Center for Biotechnology Information (NCBI) and the Olfactory Receptor Database at Yale University. BLAST searches were then performed with full-length cDNA and amino acid sequences of putative human VNO receptors that are available in the public domain, e.g., V1RL1 (Rodriguez et al.), FKSG 46, 47 and 83 (Wang). When amino acid sequences were used as probes, baits, and/or templates, TBLASTN algorithm was employed. The databases used were Human Genome Database (HGD) available at the NCBI, and ENSEMBL available at the European Biotechnology Institute (EBI). In addition to the human receptor sequences, representative mouse/rat sequences were also employed in BLAST searches in the two databases. The resulting output from all of the searches was sorted out to identify open reading frames (ORFs) that were contiguous with or without frame shifts. A large number of pseudogenes were identified in the search output. Furthermore, a sequence was deemed a potential pseudogene by the presence of one or more stop codons in a reading frame in the coding region and by the absence of a contiguous coding region in the other two reading frames.

The genomic sequences with ORFs were exported to GenTool (Double Twist, Inc.) for translation in order to obtain the putative receptor sequences. Concomitantly, searches were performed with the DNA sequences using algorithms available through Double Twist, Inc. in order to gain further information about the homology of the novel sequences, including expressed sequence tags (ESTs). The translated protein sequences were analyzed for membrane spanning domains and other characteristic features present in rodent VNO receptors and other members of the super gene family of G-protein coupled receptors (GPCR). All of the putative deduced receptor sequences were found to be approximately 300 amino acids long, a length sufficient to possess the characteristics of multiple membrane spanning receptors. They all possess a short amino and carboxy termini, a feature present in rodent subtype I and III VNO receptors. All the putative human VNO receptors identified from the current research possess seven membrane-spanning domains or seven transmembrane domains (TMs). The presence of at least two conserved cysteine residues in the GPCR family ensures proper folding of the mature protein as well as proper confirmation for the protein to be biologically active. These cysteine residues are present in Extra Cellular Loop 2 (EC2) and Extra Cellular Loop 3 (EC3) in the novel receptor sequences of hV3R1 and hV3R8. Several consensus sequences for phosyphorylation by various kinases are present in the intracellular carboxy termini. However, it is noteworthy that the GPCR-specific sequence motif at the end of TM3 (E/DRY) is absent in all of these putative VNO receptors, including rodent pheromone receptors.

II. PCR Amplification and Cloning of the Novel Vomeronasal Organ Receptors hV3R1 and hV3R8

The PCR primer shown below were synthesized based on the nucleotide sequences of Genbank Accession numbers AC004076 (HS19_{—}11261) and AC010467 (HS19_{—}11305) for hV3R₁ and hV3R8, respectively, and used to amplify specific sequences from genomic DNA. Genbank Accession numbers AC004076 and AC010467 show the raw nucleotide sequences from the genome database. The updated Genbank entry numbers are HS19_{—}11261 and HS19_{—}11305 for hV3R1 and hV3R8, respectively. HS19_{—}11261 and HS19_{—}11305 show nucleotide sequences as they code for mRNA.

PCR Primers for amplification and cloning for
hV3R1:

| | |
|---|---|
| 5′primer: | |
| GCCACCATGGTTGGAGACACATTAAAACTTCTGTCT* | (SEQ ID NO: 5) |
| 3′PRIMER: | |
| TGGCATGACAACCAGATTAGGAAAGA (SEQ ID NO: 6) | |

PCR Primers for amplification and cloning for
hV3R8:

| | |
|---|---|
| 5′primer: | |
| GCCACCATGGCCTCCCGGTATGTGGC* | (SEQ ID NO: 7) |
| 3′primer: | |
| TCTTTTCCAGGCAAAACAAAACCTG (SEQ ID NO: 8) | |

*The underlined sequence within the 5′ primers
shows homology to the consensus Kozak sequence.

The PCR conditions below were used to clone hV3R1 and dV3R8 from human genomic DNA.

100 ng of pooled human genomic DNA (5 male and 5 female DNA pooled) (Novogen) were amplified with Pfu polymerase in the following set-up:

| | |
|---|---|
| 5.0 μl | Pfu polymerase [10x] (Stratagene) |
| 1.0 μl | dNTP [10 mM each] (Sigma) |
| 1.0 μl | 5′ Primer [1.0 μg/ml] |
| 1.0 μl | 3′ Primer [1.0 μg/ml] |
| 1.0 μl | Genomic DNA [100 ng/μl] (Novagen) |
| 41.0 μl | H2O (DNA/Rnase free) |
| 50.0 μl | Total Volume |

PCR Conditions were as Follows:

10 min denaturation at 94° C.

35 cycles with 40 sec at 94° C.

2 min at 60° C. (annealing)

2 min at 72° C. (amplification)

10 min extension at 72° C.

The DNA products of the above amplification protocol were then resolved in a 1.0% agarose gel containing ethidium bromide, alongside DNA molecular weight standards (New England Biolabs Inc.), to estimate the size of the amplified DNAs. DNA of the length predicted from the sequence of Genbank entry HS19_{—}11261 and HS19_{—}11305 was excised from the gel, and then extracted from the gel and purified using the Qiaex II kit (Qiagen, Inc.), according to the manufacturer's instructions. In this manner, a specific, amplified human genomic DNA was isolated for hV3R1 and hV3R8, respectively.

Each amplified human genomic DNA was then cloned and sequenced, and the nucleotide sequence and encoded amino acid sequence compared to the corresponding sequence of Genbank entry HS19_{—}11261 and HS19_{—}11305, respectively, of known and putative pheromone receptors.

Each amplified human genomic DNA was ligated into the vector pcDNA 3.1 V5/HIS-TOPO® and the ligated DNA was transfected into competent TOP10 E. coli cells according to the manufacturer's instructions (TOPO® Cloning Kit, Invitrogen Corporation). The transformants were then selected on Luria broth agar (Invitrogen Corporation/Life Technologies) containing 100 μg of carbenecillin per ml.

Both strands of each cloned DNA, designated hV3R1 (SEQ ID NO: 1) and hV3R8 (SEQ ID NO: 3), respectively, were then sequenced by automated sequencing, using the PE DNA sequencer (Sequetech Corporation). The resulting nucleotide sequences were analyzed by alignment of the encoded amino acid sequence of GenBank entry HS19_{—}11261 for hV3R1 and by alignment of the encoded amino acid sequence GenBank entry HS19_{—}11305 for hV3R8. The results of the analysis indicated that the nucleotide sequences of the cloned human pheromone receptors hV3R1 (SEQ ID NO: 1) and hV3R8 (SEQ ID NO: 3) are identical to that predicted from the genomic DNA sequence of GenBank entry HS19_{—}11261 and HS19_{—}11305, respectively.

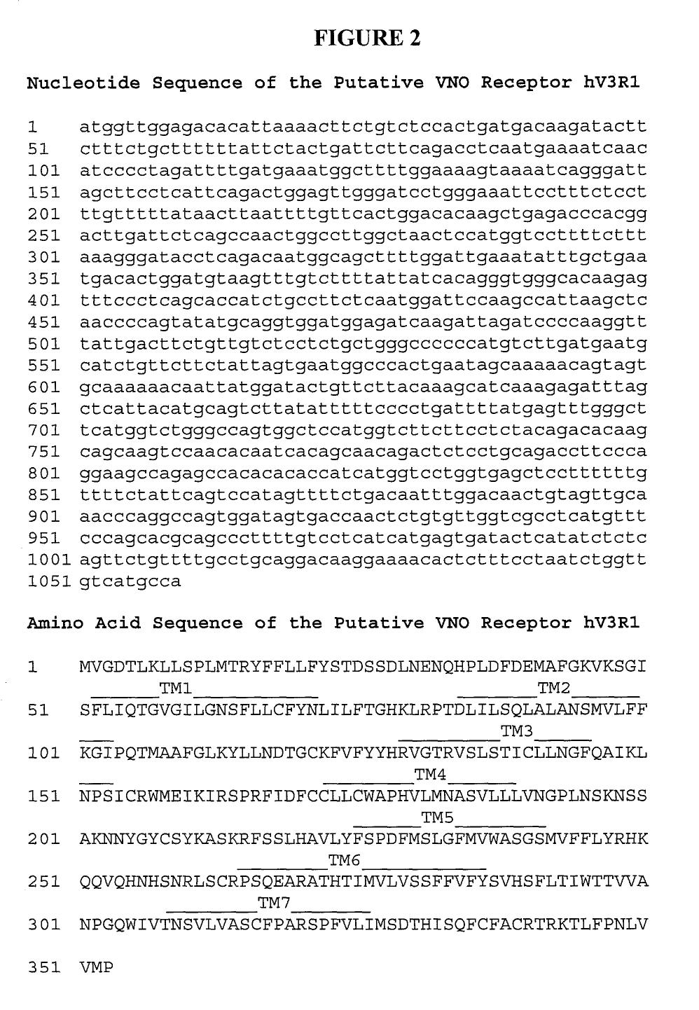

The nucleotide sequence of clone hV3R1 (SEQ ID NO: 1) predicts a polypeptide with the amino acid sequence shown in FIG. 2 (SEQ ID NO: 2). Alignment of the amino acid sequence with the amino acid sequences of putative mouse receptors and other human receptors indicated that hV3R1 shares the highest percent homology with mV3R1 and hV3R8, respectively. Allowing for conservative amino acid changes, the hV3R1 sequence is 31.7% similar to mV3R1 and 38.7% similar to hV3R8, respectively (see Table 1). Thus, hV3R1 is a novel human receptor with homology to the putative mouse receptor mV3R1 and other human pheromone receptors.

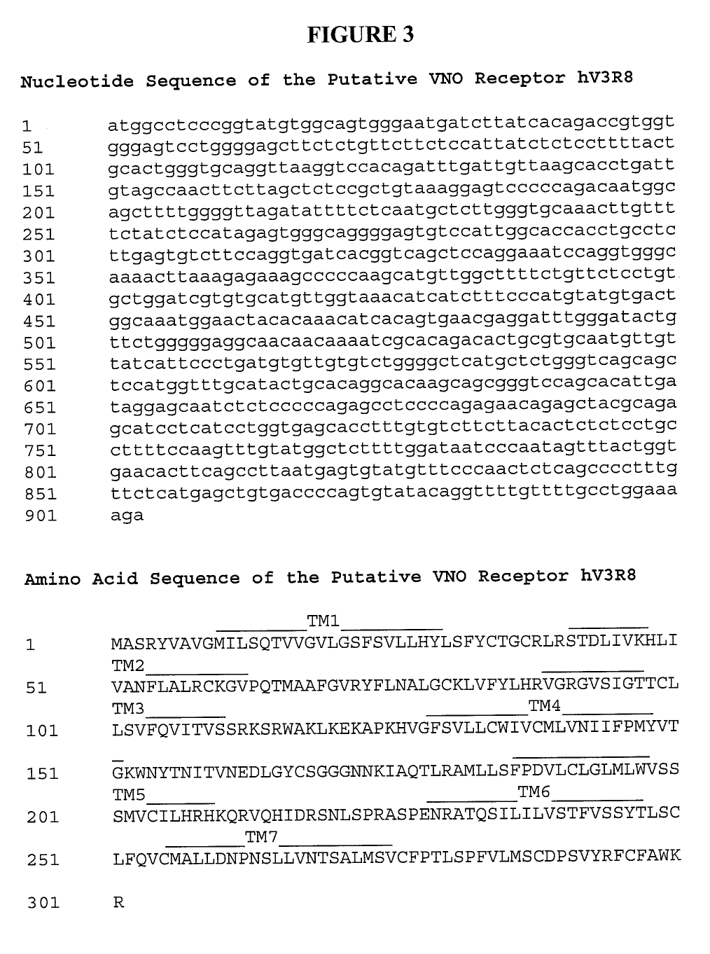

The nucleotide sequence of clone hV3R8 (SEQ ID NO: 3) predicts a polypeptide with the amino acid sequence shown in FIG. 3 (SEQ ID NO: 4). Alignment of the amino acid sequence with the amino acid sequences of putative mouse receptors and other human receptors indicated that hV3R8 shares the highest percent homology with mV3R6 and hV3R1, respectively. Allowing for conservative amino acid changes, the hV3R8 sequence is 31.7% similar to mV3R6 and 38.7% similar to hV3R1, respectively (see Table 1). Thus, hV3R8 is a novel human receptor with homology to the putative mouse receptor mV3R6 and other human pheromone receptors.

TABLE 1
AMINO ACID SEQUENCE HOMOLOGY (% HOMOLOGY)

| | hVR3R1 | hVR38 |
|---|---|---|
| mpr 1 | 25.6 | 26.7 |
| mpr 2 | 27.1 | 27.7 |
| mV3R1 | 31.7 | 29.7 |
| mV3R2 | 31.2 | 28.9 |
| mV3R3 | 28.7 | 29.9 |
| mV3R4 | 30.7 | 30.0 |
| mV3R6 | 29.5 | 31.7 |
| mV3R7 | 30.7 | 29.3 |
| mV3R8 | 31.9 | 30.0 |
| mV3R9 | 29.4 | 31.3 |
| rVN1 | 25.9 | 26.0 |
| rVN2 | 25.9 | 26.3 |
| hV3R1 | N/A | 38.7 |
| hV3R8 | 38.7 | N/A |

III. Expression Profiling of the Novel Vomeronasal Organ Receptors hV3R1 and h V3R8

An expression profile of the human pheromone receptors hV3R1 and hV3R8 was analyzed by RT-PCR in different types of human tissues (see Tables 2 and 3, respectively, vide infra). The cDNAs were purchased from either Ambion, Inc. or BioChain Institute, Inc. with the exception of human vomeronasal organ cDNA. Human vomeronasal organ cDNA was prepared from total RNA using Superscript® First Strand cDNA Synthesis Kit and oligo dT DNA primers (Invitrogen Corporation) according to the manufacturer's instructions. The PCR conditions for expression profiling are shown below.

The primers used for expression profiling for hV3R1 were as follows:

(SEQ ID NO: 9)

| | |
|---|---|
| 5′ Primer: | TCTTCCTCTACAGACACAAGCAGCAAGT |

(SEQ ID NO: 10)

| | |
|---|---|
| 3′ Primer: | GGCATGACAACCAGATTAGGAAAGAGTGTTTTCCTT |

The primers used for expression profiling for hV3R8 were as follows:

| | |
|---|---|
| 5′ Primer: | |
| TGTTATCATTCCCTGATGTGTTGTGTCTGGGG | (SEQ ID NO: 11) |
| 3′ Primer: | |
| TCCAGGCAAAACAAAACCTGTATACACTGGGGT | (SEQ ID NO: 12) |

PCR conditions were as follows

10 min denaturation at 96° C.

35 cycles of

30 sec at 96° C.

30 sec at 60° C. (annealing)

30 sec at 68° C. (amplification)

10 min extension at 68° C.

The expression profiling results are shown in Tables 2 and 3 (infra). hV3R1 expression was found in all brain tissues tested, testis, retina and male and female VNO. hV3R8 expression was found in amygdala, olfactory tissues, and male and female VNO.

TABLE 2
RT-PCR Expression Profile of hV3R1 mRNA in Human Tissue

| Human Tissue | Positive | Negative |
|---|---|---|
| Testis | ✓ | |
| Lung | | ✓ |
| Liver | | ✓ |
| Kidney | | ✓ |
| Amygdala | ✓ | |
| Olfactory | ✓ | |
| Cerebral cortex | ✓ | |
| Occipital lobe | ✓ | |
| Frontal lobe | ✓ | |
| Hippocampus | ✓ | |
| Parietal lobe | ✓ | |
| Thalamus | ✓ | |
| Cerebellum (left) | ✓ | |
| Temporal lobe | ✓ | |
| Cerebral peduncle | ✓ | |
| Postcentral gyrus | ✓ | |
| Diencephalon | ✓ | |
| Pons | ✓ | |
| Corpus callosum | ✓ | |
| Heart | | ✓ |
| Spleen | | ✓ |
| Prostate | | ✓ |
| Ovary | | ✓ |
| Male VNO | ✓ | |
| Female VNO | ✓ | |
| Skin | | ✓ |
| Retina | ✓ | |

TABLE 3
RT-PCR Expression Profile of hV3R8 mRNA in Human Tissue

| Human Tissue | Positive | Negative |
|---|---|---|
| Testis | | ✓ |
| Lung | | ✓ |
| Liver | | ✓ |
| Kidney | | ✓ |
| Amygdala | ✓ | |
| Olfactory | ✓ | |
| Cerebral cortex | | ✓ |
| Occipital lobe | | ✓ |
| Frontal lobe | | ✓ |
| Hippocampus | | ✓ |
| Parietal lobe | | ✓ |
| Thalamus | | ✓ |
| Cerebellum (left) | | ✓ |
| Temporal lobe | | ✓ |
| Cerebral peduncle | | ✓ |
| Postcentral gyrus | | ✓ |
| Diencephalon | | ✓ |
| Pons | | ✓ |
| Corpus callosum | | ✓ |
| Heart | | ✓ |
| Spleen | | ✓ |
| Prostate | | ✓ |
| Ovary | | ✓ |
| Male VNO | ✓ | |
| Female VNO | ✓ | |
| Skin | ✓ | |
| Retina | ✓ | |

Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the claims. All publications, patents, and other reference materials referred to herein are incorporated herein by reference.

What is claimed is:

1. An isolated nucleic acid molecule encoding a polypeptide of SEQ ID NO: 2 or SEQ ID NO: 4, or polypeptide fragment thereof, wherein said polypeptide or polypeptide fragment mediates a detectable physiological or behavioral effect in mammals.

2. An isolated nucleic acid molecule of claim 1, wherein said nucleic acid molecule is a DNA or RNA.

3. An isolated nucleic acid molecule that hybridizes under high stringency conditions to a nucleic acid molecule of claim 1.

4. An isolated nucleic acid molecule that is complementary to a nucleic acid molecule of claim 1.

5. An oligonucleotide probe or primer comprising about 7 to about 50 nucleotides of a nucleic acid of claim 1.

6. A composition comprising an isolated nucleic acid molecule of claim 1 and a suitable adjuvant.

7. A vector comprising an isolated nucleic acid molecule of claim 1, wherein said vector expresses in cells the polypeptide encoded by said nucleic acid molecule.

8. An isolated cell comprising a vector of claim 7, wherein said cell is a mammalian, bacterial, insect or yeast cell.

9. An isolated cell of claim 8, wherein said polypeptide is expressed as a cell-surface receptor.

10. An isolated nucleic acid molecule which encodes a polypeptide of SEQ ID NO: 2 or SEQ ID NO: 4.

11. An isolated nucleic acid molecule comprising the nucleotide sequence of SEQ ID NO: 1 or SEQ ID NO: 3.

12. An isolated polypeptide comprising the amino acid sequence set forth in SEQ ID NO: 2 or SEQ ID NO: 4, or fragment thereof, wherein said polypeptide or fragment thereof mediates a detectable physiological or behavioral effect in mammals.

13. A polypeptide of claim 12 which is a human polypeptide.

14. A composition comprising an isolated polypeptide of claim 12 and a suitable adjuvant.

15. An isolated polyclonal or monoclonal antibody that specifically binds to a polypeptide of claim 12.

16. A composition comprising an isolated polyclonal or monoclonal antibody of claim 15 and a suitable adjuvant.

17. An isolated polypeptide comprising the amino acid sequence of SEQ ID NO: 2 or SEQ ID NO: 4.

18. A method for mediating a physiological disorder comprising administering to a subject an effective amount of a composition comprising an isolated nucleic acid molecule of claim 1 and inducing expression thereof, wherein said subject is in need of treatment for diseases or disorders selected from the group consisting of infertility; disorders related to contraception; disorders related to hormonal regulation, sexual dysfunction, erectile dysfunction; neurological-, psychiatric-, liver-, spleen disorders; cardiovascular disorders; cancer; and known or unknown disorders related to the aberrant expression of the human pheromone receptor hV3R1 or hV3R8.

19. A method for mediating a physiological disorder comprising administering an effective amount of an isolated polypeptide of claim 12 to a subject, wherein said subject is in need of treatment for diseases or disorders selected from the group consisting of infertility; disorders related to contraception; disorders related to hormonal regulation, sexual dysfunction, erectile dysfunction; neurological-, psychiatric-, liver-, spleen disorders; cardiovascular disorders; cancer; and known or unknown disorders related to the aberrant expression of the human pheromone receptor hV3R1 or hV3R8.

20. A method for mediating a physiological disorder comprising administering an isolated polyclonal or monoclonal antibody of claim 16, wherein said subject is in need of treatment for diseases or disorders selected from the group consisting of infertility; disorders related to contraception; disorders related to hormonal regulation, sexual dysfunction, erectile dysfunction; neurological-, psychiatric-, liver-, spleen-disorders; cardiovascular disorders; cancer; and known or unknown disorders related to the aberrant expression of the human pheromone receptor hV3R1 or hV3R8, said method comprising administering to said subject an effective amount of said composition by topical, oral, inhalable, subcutaneous or intramuscular administration.

21. A method for detecting a target nucleic acid molecule in a sample using as a probe an at least 7 nucleotide contiguous segment in length of an isolated nucleic acid molecule of claim 1, said method comprising the steps of:
a) exposing said probe to a sample, under conditions where the probe hybridizes under high, medium or low stringency conditions to said target nucleic acid molecule in the sample; and
b) detecting said target nucleic acid molecule hybridized to said probe.

22. The method of claim 21, wherein said probe is immobilized or in solution.

23. A method for detecting a polypeptide in a sample using as a probe a polypeptide of claim 12, said method comprising the steps of:
a) exposing said probe to a sample, under conditions where the probe specifically binds to said polypeptide in the sample, and
b) detecting said polypeptide bound to the probe.

24. The method of claim 23, wherein said probe is immobilized or in solution.

25. A method of identifying molecules that mediate a detectable physiological or behavioral response in mammals by screening for the interaction of a test molecule with a polypeptide molecule of claim 12.

26. A transgenic animal that expresses the polypeptide encoded by the amino acid sequence of SEQ ID NO: 2 or SEQ ID NO: 4.


SEQUENCE LISTING (12 sequences)

SEQ ID NO: 1 - DNA, 1059 bp, Homo sapiens
atggttggagacacattaaaacttctgtctccactgatgacaagatacttctttctgcttttttattctactgattcttcagacctcaatgaaaatcaacatcccctagattttgatgaaatggcttttggaaaagtaaaatcagggattagcttcctcattcagactggagttgggatcctgggaaattcctttctcctttgtttttataacttaattttgttcactggacacaagctgagacccacggacttgattctcagccaactggccttggctaactccatggtccttttctttaaagggatacctcagacaatggcagcttttggattgaaatatttgctgaatgacactggatgtaagtttgtcttttattatcacagggtgggcacaagagtttccctcagcaccatctgccttctcaatggattccaagccattaagctcaaccccagtatatgcaggtggatggagatcaagattagatccccaaggtttattgacttctgttgtctcctctgctgggccccccatgtcttgatgaatgcatctgttcttctattagtgaatggcccactgaatagcaaaaacagtagtgcaaaaaacaattatggatactgttcttacaaagcatcaaagagatttagctcattacatgcagtcttatatttttcccctgattttatgagtttgggcttcatggtctgggccagtggctccatggtcttcttcctctacagacacaagcagcaagtccaacacaatcacagcaacagactctcctgcagaccttcccaggaagccagagccacacacaccatcatggtcctggtgagctccttttttgttttctattcagtccatagttttctgacaatttggacaactgtagttgcaaacccaggccagtggatagtgaccaactctgtgttggtcgcctcatgtttcccagcacgcagcccttttgtcctcatcatgagtgatactcatatctctcagttctgttttgcctgcaggacaaggaaaacactctttcctaatctggttgtcatgcca

SEQ ID NO: 2 - protein, 353 aa, Homo sapiens
MVGDTLKLLSPLMTRYFFLLFYSTDSSDLNENQHPLDFDEMAFGKVKSGISFLIQTGVGILGNSFLLCFYNLILFTGHKLRPTDLILSQLALANSMVLFFKGIPQTMAAFGLKYLLNDTGCKFVFYYHRVGTRVSLSTICLLNGFQAIKLNPSICRWMEIKIRSPRFIDFCCLLCWAPHVLMNASVLLLVNGPLNSKNSSAKNNYGYCSYKASKRFSSLHAVLYFSPDFMSLGFMVWASGSMVFFLYRHKQQVQHNHSNRLSCRPSQEARATHTIMVLVSSFFVFYSVHSFLTIWTTVVANPGQWIVTNSVLVASCFPARSPFVLIMSDTHISQFCFACRTRKTLFPNLVVMP

SEQ ID NO: 3 - DNA, 903 bp, Homo sapiens
atggcctcccggtatgtggcagtgggaatgatcttatcacagaccgtggtgggagtcctggggagcttctctgttcttctccattatctctccttttactgcactgggtgcaggttaaggtccacagatttgattgttaagcacctgattgtagccaacttcttagctctccgctgtaaaggagtcccccagacaatggcagcttttggggttagatattttctcaatgctcttgggtgcaaacttgttttctatctccatagagtgggcaggggagtgtccattggcaccacctgcctcttgagtgtcttccaggtgatcacggtcagctccaggaaatccaggtgggcaaaacttaaagagaaagcccccaagcatgttggcttttctgttctcctgtgctggatcgtgtgcatgttggtaaacatcatctttcccatgtatgtgactggcaaatggaactacacaaacatcacagtgaacgaggatttgggatactgttctgggggaggcaacaacaaaatcgcacagacactgcgtgcaatgttgttatcattccctgatgtgttgtgtctggggctcatgctctgggtcagcagctccatggtttgcatactgcacaggcacaagcagcgggtccagcacattgataggagcaatctctcccccagagcctccccagagaacagagctacgcagagcatcctcatcctggtgagcacctttgtgtcttcttacactctctcctgccttttccaagtttgtatggctcttttggataatcccaatagtttactggtgaacacttcagccttaatgagtgtatgtttcccaactctcagcccctttgttctcatgagctgtgaccccagtgtatacaggttttgttttgcctggaaaaga

SEQ ID NO: 4 - protein, 301 aa, Homo sapiens
MASRYVAVGMILSQTVVGVLGSFSVLLHYLSFYCTGCRLRSTDLIVKHLIVANFLALRCKGVPQTMAAFGVRYFLNALGCKLVFYLHRVGRGVSIGTTCLLSVFQVITVSSRKSRWAKLKEKAPKHVGFSVLLCWIVCMLVNIIFPMYVTGKWNYTNITVNEDLGYCSGGGNNKIAQTLRAMLLSFPDVLCLGLMLWVSSSMVCILHRHKQRVQHIDRSNLSPRASPENRATQSILILVSTFVSSYTLSCLFQVCMALLDNPNSLLVNTSALMSVCFPTLSPFVLMSCDPSVYRFCFAWKR

SEQ ID NO: 5 - DNA, 36 bp, Artificial Sequence
  Primer used for amplification and cloning.
gccaccatggttggagacacattaaaacttctgtct

SEQ ID NO: 6 - DNA, 26 bp, Artificial Sequence
  Primer used for amplification and cloning.
tggcatgacaaccagattaggaaaga

SEQ ID NO: 7 - DNA, 26 bp, Artificial Sequence
  Primer used for amplification and cloning.
gccaccatggcctcccggtatgtggc

SEQ ID NO: 8 - DNA, 25 bp, Artificial Sequence
  Primer used for amplification and cloning.
tcttttccaggcaaaacaaaacctg

SEQ ID NO: 9 - DNA, 28 bp, Artificial Sequence
  Primer used for expression profiling.
tcttcctctacagacacaagcagcaagt

SEQ ID NO: 10 - DNA, 36 bp, Artificial Sequence
  Primer used for expression profiling.
ggcatgacaaccagattaggaaagagtgttttcctt

SEQ ID NO: 11 - DNA, 32 bp, Artificial Sequence
  Primer used for expression profiling.
tgttatcattccctgatgtgttgtgtctgggg

SEQ ID NO: 12 - DNA, 33 bp, Artificial Sequence
  Primer used for expression profiling.
tccaggcaaaacaaaacctgtatacactggggt